(19) United States
(12) Patent Application Publication
(10) Pub. No.: US 2003/0119015 A1
(43) Pub. Date: Jun. 26, 2003

(54) Methods for nucleic acid analysis

(75) Inventors: Kelly A. Frazer, San Mateo, CA (US); Nila Patil, Woodside, CA (US); John Sheehan, Mountain View, CA (US)

Correspondence Address: BEYER WEAVER & THOMAS LLP, P.O. BOX 778, BERKELEY, CA, 94704-0778, US

(73) Assignee: Perlegen Sciences, Inc.

(21) Appl. No.: 10/142,364

(22) Filed: May 8, 2002

Related U.S. Application Data
(60) Provisional application No. 60/337,567, filed on Nov. 30, 2001.
(60) Provisional application No. 60/337,094, filed on Dec. 6, 2001.
(60) Provisional application No. 60/357,569, filed on Feb. 13, 2002.
(60) Provisional application No. 60/371,862, filed on Apr. 10, 2002.

(30) Foreign Application Priority Data
May 10, 2001 (WO) PCT/US01/15139

(51) Int. Cl. C12Q 1/68; G06F 19/00; G01N 33/48; G01N 33/50; C12P 19/34

(52) U.S. Cl. 435/6; 702/20; 435/91.2

(57) ABSTRACT

The present invention provides methods for determining sequence similarity (conserved sequences) between nucleic acids from a first organism and nucleic acids from a second, different organism without having to know a priori the nucleic acid sequence from the second, different organism. The first nucleic acid can be from any organism where the sequence of the nucleic acid is known and the second nucleic acid can be from any organism. The method involves determining which bases from the second nucleic acid are identical to the first nucleic acid, and allows one to determine the sequence of portions of the second nucleic acid. The invention is useful for identifying putative functional regions or putative organism-sequences in a genome.

### RELATED APPLICATIONS

This application claims priority to PCT/US01/15139, filed May 10, 2001; U.S. Ser. No. 09/972,595, filed Oct. 5, 2001, which claims priority to provisional application U.S. S No. 60/284,436, filed Apr. 18, 2001 and patent application U.S. Ser. No. 09/853,113, filed May 10, 2001; patent application U.S. Ser. No. 09/972,595, filed Oct. 5, 2001; provisional application U.S. S No. 60/337,567, filed Nov. 30, 2001; provisional application U.S. S No. 60/337,094, filed Dec. 6, 2001; provisional application U.S. S No. 60/357,569, filed Feb. 13, 2002 and provisional application U.S. S No. 60/371,862, filed Apr. 10, 2002, each of which is incorporated by reference in its entirety for all purposes.

### BACKGROUND OF THE INVENTION

The sequence of the human genome is now available. It is estimated that only 5% of the human genome contains coding regions. The value of identifying coding sequence is clear as variation in coding sequences can have a direct impact on the encoded protein and the functionality of the gene; thus, there is a tremendous effort in the genomics community to identify such coding sequences. However, in addition to coding sequences, there are non-coding sequences in the genome that have great importance in determining gene function. These important non-coding sequences contain regulatory regions, such as promoters, enhancers, ribosome binding sites, transcription termination sites and the like. Sifting through the 95% of the genome comprised of non-coding sequences to identify the non-coding elements with biological importance is an imposing challenge. Therefore, methods to rapidly identify functional, non-coding sequences in the human genome or the genome of any organism are needed.

### SUMMARY OF THE INVENTION

The present invention provides methods for analyzing a plurality of nucleic acid sequences to identify sequences that are evolutionarily conserved between two species; to identify sequences that are transcribed; to identify sequences that have been rearranged between two species since their last shared common ancestor; to analyze tumor and other somatic cell rearrangements; or to identify differences between regulatory elements (including but not limited to those involved in transcription).

The method comprises collecting a plurality of hybridization intensities wherein each of said intensities reflects the hybridization of one of a plurality of probes from a first nucleic acid sequence from a first organism to a sample nucleic acid from a second organism, wherein said probes are complementary and non-complementary to a known nucleic acid sequence from said first organism, wherein said probes are arrayed on a substrate and wherein each detection probe is at a known location on said substrate; identifying bases of said plurality of probes according to said hybridization intensities; and calculating an identity index between said first nucleic acid sequence from said first organism and said sample nucleic acid from said second organism.

According to one aspect of the invention, the identity index is calculated by determining a percentage of similarity between sub-regions of said nucleic acids from said first organism and said nucleic acids from said second organism. The sub-regions preferably are overlapping, moving windows of base pairs across said nucleic acid sequence from a first organism. In one embodiment, the windows are between about 20 base pairs and 150 base pairs and the overlap of said windows is between about 5 base pairs and about 75 base pairs. In another embodiment, the windows are about 30 base pairs with an overlap of about 10 base pairs.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1 is a schematic of the detection and analysis of evolutionarily conserved sequences on human chromosome 21.

FIG. 2 shows a chromosome 21 reference sequence tiled as 25-mer oligonucleotides (probes).

FIG. 3 shows an enlarged view of a human 21q array hybridized with syntenic dog BAC DNA (top).

FIG. 4 shows a CONSEQ plot of conserved regions identified by hybridization with syntenic dog sequences for a 26-kb interval on chromosome 21.

FIG. 5 shows scans of four identical substrate-bound oligonucleotide arrays with probes based on the human genomic sequence from chromosome 21 hybridized with (A) human, (B) gorilla, (C) chimpanzee and (D) macaque genomic DNA samples.

FIG. 6 shows CONSEQ plots of conserved regions identified by hybridization with orthologous dog and mouse sequences for a 100-kb interval on chromosome 21 (bottom two plots). The annotations in these plots are the same as described for FIG. 3.

FIG. 7 is a block diagram of a computer system that may be used to implement various aspects of this invention.

FIG. 8 shows an analysis of syntenic human and chimpanzee LR-PCR products for deletions and insertions. In Panel A, the lengths of syntenic human (H) and chimpanzee (C) LR-PCR products are compared by gel electrophoresis. In Panel B, the human and chimpanzee LCR-PCR products shown in (A) were hybridized to the 21q arrays and their percent conformances (vertical axis), which is a measure of their similarity, were plotted relative to their position in the human reference sequence (horizontal axis). Each tick mark in the scale represents a 1 kb interval. The sequence positions of the PCR products in (A), and (C) are indicated by horizontal lines. In Panel C, paired PCR primers designed to the external boundaries of the deletions in LRPCR products 1-5 in (A) as shown in (B), were used to amplify human and chimpanzee DNA.

FIG. 9 shows the relative sizes of the syntenic human (H), chimpanzee (C), and orangutan (O) LR-PCR products are used to determine whether the rearrangement occurred in the human or chimpanzee genome and if it was an insertion or deletion event.

FIG. 10 shows the distribution of 57 human-chimpanzee rearrangements (black) and 76 human specific LR-PCR products (red) in 250 kb adjacent intervals along the length of human chromosome 21. The green bar denotes the position on chromosome 21 (about 10.0 to 11.4 Mb from the centromeric end) containing an increased number of rearrangements and/or an increased amount of sequence divergence.

### DETAILED DESCRIPTION OF EXAMPLE EMBODIMENTS

I. Definitions

As used herein the specification, “a” or “an” may mean one or more. As used herein in the claim(s), when used in conjunction with the word “comprising”, the words “a” or “an” may mean one or more than one. As used herein “another” may mean at least a second or more.

Bind(s) substantially refers to complementary hybridization between a probe nucleic acid and a target nucleic acid and embraces minor mismatches that can be accommodated by reducing the stringency of the hybridization media to achieve the desired detection of the target polynucleotide sequence.

Complementary refers to a single-stranded nucleotide sequence having a sufficient number of pairing bases such that it specifically (non-randomly) hybridizes to another single stranded nucleotide sequence with consequent hydrogen bonding.

The phrase “hybridizing specifically to” refers to the binding, duplexing, or hybridizing of a molecule only to a particular nucleotide sequence under stringent conditions when that sequence is present in a complex mixture (e.g., total cellular) DNA or RNA.

Massively parallel screening refers to the simultaneous screening of at least about 100, preferably about 1000, more preferably about 10,000 and most preferably about 1,000,000 different nucleic acid hybridizations.

A nucleic acid is a deoxyribonucleotide or ribonucleotide polymer in either single- or double-stranded form, including known analogs of natural nucleotides unless otherwise indicated.

An oligonucleotide is a single-stranded nucleic acid ranging in length from 2 to about 500 bases.

A probe is a nucleic acid capable of binding to a target nucleic acid of complementary sequence through one or more types of chemical bonds, usually through complementary base pairing, usually through hydrogen bond formation. A nucleic acid probe may include natural (i.e. A, G, C, or T) or modified bases (e.g., 7-deazaguanosine, inosine). In addition, the bases in a nucleic acid probe may be joined by a linkage other than a phosphodiester bond, so long as it does not interfere with hybridization. Thus, nucleic acid probes may be peptide nucleic acids in which the constituent bases are joined by peptide bonds rather than phosphodiester linkages.

Putative functional regions include known functional regions and also regions that meet the criteria described herein for functional regions but which need further verification or testing to demonstrate they are functional regions.

Putative organism-differentiating regions designate regions which are known organism-differentiating regions and those which match the criteria specified herein for organism-differentiating regions, but which need further testing to confirm or verify that they are organism-differentiating regions.

Specific hybridization refers to the binding, duplexing, or hybridizing of a molecule only to a particular nucleotide sequence under stringent conditions when that sequence is present in a complex mixture (e.g., total cellular) DNA or RNA. Stringent conditions are conditions under which a probe hybridizes to its target subsequence, but to no other sequences. Stringent conditions are sequence-dependent and are different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. (As the target sequences are generally present in excess, at Tm, 50% of the probes are occupied at equilibrium). Typically, stringent conditions include a salt concentration of at least about 0.01 to 1.0 M Na ion concentration (or other salts) at pH 7.0 to 8.3 and the temperature is at least about 30C for short probes (e.g., 10 to 50 nucleotides). Stringent conditions can also be achieved with the addition of destabilizing agents such as formamide. For example, conditions of 5×SSPE (750 mM NaCl, 50 mM sodium phosphate, 5 mM EDTA, pH 7.4) and a temperature of 25-30° C. are suitable for allele-specific probe hybridizations. A perfectly matched probe has a sequence perfectly complementary to a particular target sequence. The test probe is typically perfectly complementary to a portion (subsequence) of the target sequence. The term “mismatch probe” refers to probes whose sequence is deliberately selected not to be perfectly complementary to a particular target sequence. Although the mismatch(es) may be located anywhere in the mismatch probe, terminal mismatches are less desirable as a terminal mismatch is less likely to prevent hybridization of the target sequence. Thus, probes are often designed to have the mismatch located at or near the center of the probe such that the mismatch is most likely to destabilize the duplex with the target sequence under the test hybridization conditions.

Target nucleic acid refers to a nucleic acid (often derived from a biological sample), to which the oligonucleotide probe is designed to specifically hybridize. It is either the presence or absence of the target nucleic acid that is to be detected, or the amount of the target nucleic acid that is to be quantified. The target nucleic acid has a sequence that is complementary to the nucleic acid sequence of the corresponding probe directed to the target. The term target nucleic acid may refer to the specific subsequence of a larger nucleic acid to which the probe is directed or to the overall sequence (e.g., gene or mRNA) whose expression level it is desired to detect. The difference in usage will be apparent from context.

Reference will now be made in detail to the preferred embodiments of the invention. While the invention will be described in conjunction with preferred embodiments, it should be understood that such embodiments are not intended to limit the invention to these embodiments. On the contrary, the invention is intended to cover alternatives, modifications and equivalents which are included within the spirit and scope of the invention. For example, the invention will be described by referring to embodiments providing methods, compositions, data analysis systems and computer program products for discovering functional regions in a genome. However, the methods, compositions, computational analysis and computer program products may be useful for analyzing the sequences of other biological molecules, particularly those useful for comparing sequences when one sequence is known and the other is not. In addition, one skilled in the art recognizes that the term “species” is an artificial designation for organisms, and that the present invention can be applied to make sequence comparisons of organisms that are in the same species but in different strains, organisms that are hybrids, or organisms that are related to each other genetically in other ways. Further, although human sequence is used as an example of a reference or known sequence useful in the present invention, the present invention should not be limited to use with human sequence. The reference or known sequence can be any known sequence from any organism. One skilled in the art recognizes that when first substrate and second substrate are referenced herein that both the first and second substrates could be different substrates or that a single substrate is used in both cases. In the later case, after use of the substrate as the first substrate, the conditions on the substrate are changed such that the sequences hybridized on the first use are removed and the substrate is then used as the second substrate.

All patents and publications mentioned in the specification are indicative of the level of those skilled in the art to which the invention pertains. All patents and publications are herein incorporated by reference to the same extent as if each individual publication was specifically and individually indicated to be incorporated by reference.

II. Overview

Generally, this invention relates to biological and computational methods for identifying regions that have been conserved through human evolution or otherwise annotating regions of the human genome. More specifically, the present invention provides methods for recognizing functional sequences in a human genome—both in coding regions and in non-coding regions—by employing techniques that allow the comparison of the genomic sequence of a human to another organism to identify nucleic acid sequences that are conserved between the two organisms. The second organism may be a human or from a different species. Cross-species sequence comparisons are powerful methods for decoding genomic information because functional elements are conserved through evolution whereas nonfunctional sequences drift. The present invention particularly is powerful as it allows such comparison without having to know the nucleic acid sequence of both organisms as is necessary in prior art comparison methods—in the present invention, knowledge of the nucleic acid sequence of only one of the organism is necessary.

More specifically, computer-based methods and systems and media for evaluating biological sequences from a plurality of organisms are provided. In accordance with this invention, one or more biological sequences from a first organism are compared with those from a second organism in certain ways such that relevant features can be extracted. Typically, the biological sequences derived from the first species will be nucleic acids which have been immobilized on a substrate (the detection probes). The biological sequences from the second organism are then contacted with the substrate and the amount and position of hybridization that takes place are evaluated.

The invention further provides methods for analyzing the hybridization data for the identification of sequences that are evolutionarily conserved between two species; the identification of sequences that are transcribed; the identification of sequences that have been rearranged between two species since their last shared common ancestor; the analysis of tumor and other somatic cell rearrangements; and the identification of differences between regulatory elements (including but not limited to those involved in transcription). These methods are based on the analysis of an identity index between sub-regions of the nucleic acids from the first organism and the nucleic acids from the second organism or the third organism. The identity index may be calculated by determining a percentage of similarity between sub-regions of the nucleic acids from the first organism and the nucleic acids from the second or third organism. In addition, the methods may designate the sub-regions as overlapping, moving windows of base pairs across the nucleic acid sequence from a first organism, wherein the windows are between about 20 base pairs and 150 base pairs, the overlap of the windows is between about 5 base pairs and about 75 base pairs. One application of the present invention designated windows of 30 base pairs and an overlap of 10 base pairs. As described further below, the parameters may vary depending on the data set.

III. Selection of Organisms

The methods of the invention provide for comparing the nucleic acid from a first organism to that of a second organism. The sequence of the second organism may, or may not, be known. According to one embodiment, the nucleic acid sequence of the second organism is not previously known, but rather has been determined from a hybridization experiment as described further below. However, as one of skill in the art will appreciate, the analysis algorithm described herein can be applied to the comparison of any two sequences regardless of how they were generated.

The organisms may be of the same or of different species. In general, organisms that diverged evolutionarily over about 120 million years ago share genomic similarity in exonic regions. In contrast, organisms that diverged evolutionarily between about 60 and about 120 million year ago share genomic similarity in both exonic regions and regulatory elements whereas organisms that diverged less than about 60 million years ago share genomic sequence similarity in genomic regions other than exonic regions and regulatory elements. Thus, regions of sequence similarity are more or less informative depending on the relatedness of the two organisms compared.

For example, if two organisms diverged evolutionarily between about 60 million and about 120 million years ago, identifying sequences conserved between the organisms would identify putative functional regions (coding and non-coding functional regions) in the genomes of the organisms. On the other hand, if two organisms diverged evolutionarily less than about 60 million years ago, many sequences may be conserved due to insufficient divergence time. Thus, identifying sequences that are not conserved between the organisms—regions of sequence divergence—would identify putative organism-differentiating and rapidly evolving regions.

In another aspect of the invention, the first organism can be any organism where a sequence of DNA is known and the second organism can be any other organism where there is greater than about 60 million years and less than about 120 million years of evolutionary divergence between the first organism and the second organism. Preferably, the first organism is a human, and the second species is a non-human mammal where there is greater than about 60 million years and less than about 120 million years of evolutionary divergence between the human and the non-human mammal.

In another embodiment, the genomic sequence of a first organism is compared with the genomic sequence of a second organism where there is less than about 60 million years of evolutionary divergence between the first organism and the second organism. In one application, the first organism is a human, and the second organism is a gorilla; however, the present invention provides that the first organism can be any organism where a sequence of DNA is known and the second organism can be any other closely-related organism.

In another aspect, regions of a genome that are conserved between a plurality of organisms are determined. Sequences that tend to be conserved between a plurality of organisms are likely to be conserved due to functionality of the sequence, and not be conserved due to chance or insufficient divergence time. Thus, sequences between a first organism (where the nucleic acid sequence is known) and a second organism (where the nucleic acid sequence is not known) are compared, and then between the first organism and a third organism (where the nucleic acid sequence is not known), where there is greater than about 60 million years and less than about 120 million years of evolutionary divergence between the first organism and at least one of the other organisms.

Sequences that tend to be conserved between all three organisms are likely to be conserved due to functionality of the sequence, and not be conserved due to insufficient divergence time. Accordingly, comparisons can be done between any number of organisms to achieve greater accuracy. In addition, if one of the other organisms has greater than 60 million years of evolutionary divergence from the first organism, and a third organism has less than 60 million years of evolutionary divergence from the first organism, it is possible to detect sequences which are being conserved and sequences that are evolving rapidly. Sequences that are evolving rapidly have greater than average sequence divergence between one organism and the other and are difficult to detect, i.e., less sequence similarity; but what is similar is important. Yet these rapidly evolving sequences are scientifically and practically very interesting.

In one aspect of the present invention, the first nucleic acid is derived from a human, and the second nucleic acid is derived from another animal species. Use of human sequence at this time makes sense as it is one of the few complete genomes that has been sequenced to date; however, the first nucleic acid can be from any organism where the sequence of the nucleic acid is known and the second nucleic acid can be from any organism.

In another aspect, the first nucleic acid is derived from a first human and the second nucleic acid is derived from a second human.

IV. Preparation of Target

The target polynucleotide is usually isolated from a tissue sample from the organism of interest. If the target is genomic DNA, the sample may be from any tissue (except red blood cells). These sources are also suitable if the target is RNA. Methods for isolating genomic DNA are known in the art (see, e.g., Sambrook, et al., Molecular Cloning: A Laboratory Manual (1989), 2d Ed., Cold Spring Harbor, N.Y.). For closely-related organisms, typically the genomic DNA sample is prepared by extraction of genomic DNA from the second organism, followed by long range amplification of the DNA by the polymerase chain reaction using primers based on the reference sequence. For less related organisms, it may be necessary to sub-clone portions of the genomic DNA of the second organism into a cloning vector before amplification.

In certain embodiments of the present invention, the DNA for the nucleic acid sample is amplified. Amplification methods are well known in the art, and the method selected generally depends on the size of the regions to be amplified. If, for example, the regions to be amplified are contained in vectors or artificial chromosomes, PCR methods known in the art can be employed. If the DNA to be amplified is genomic DNA, long range PCR methods preferentially are employed. In order to amplify genomic DNA, PCR primers must be designed for the amplification reaction. Primers used for the amplification reaction are designed in the following way: a given sequence, usually the reference sequence, is fed into a software program called “Repeat Masker” which recognizes sequences that are repeated in the genome (e.g., Alu and Line elements) (A. F. A. Smit and P. Green, http://www.genome.washington.edu/ uwgc/analysistools/repeatmask.htm.) The repeated sequences are “masked” by the program by substituting the specific nucleotides of the sequence (A, T, G or C) with “Ns”. The sequence output after the repeat mask substitution can then be analyzed by a commercially available primer design program (for example, Oligo 6.23 or PrimerSelect) to select primers that meet criteria appropriate for the size of the regions to be amplified and the reaction conditions chosen. For example, primer criteria used might dictate that the primers have a length of greater than 30 nucleotides, melting temperatures of over 65° C., and amplify at least 3,000 bps of the genome. In a preferred embodiment, each primer pair is tested by performing two PCR reactions, one with genomic DNA matching the reference sequence (that is, nucleic acid isolated from the first species) and the other with target DNA. This test is performed to determine whether the primer pair produces a single clear amplified fragment visible by agarose gel electrophoresis and ethidium bromide staining. See Attorney Docket No. 1011U1 filed Jan. 9, 2002, entitled “Algorithms for selection of primer pairs”, U.S. Ser. No. 10/042,406.

PCR reactions may be performed by methods known in the art. Such methods are described in laboratory manuals such as Sambrook, et al., Molecular Cloning: A Laboratory Manual (1989), 2d Ed., Cold Spring Harbor, N.Y. Long distance PCR is described in, for example, product literature from, e.g., Roche (Expand Long Template PCR System), or Takara Shuzo Co., Ltd. (TaKaRa LA Taq), as described in U.S. Pat. No. 5,512,462 to Cheung, or as described in (Attorney Docket No. 1011U1D1), filed Jan. 9, 2002, entitled “Methods for amplification of nucleic acids”, U.S. Ser. No. 10/042,492, all of which are incorporated in their entirety herein by reference. In addition, more than one target region can be amplified simultaneously by multiplex PCR in which multiple paired primers are used in a single amplification reaction. The target can be labeled at one or more nucleotides during or after amplification. Many labels are known in the art, including luminescent labels, radioactive labels, and light scattering labels. Preferably, the label is a luminescent label, such as fluorescent, chemiluminescent, bioluminescent or colorimetric labels. The target preferably is fragmented before hybridization with the array to reduce or eliminate the formation of secondary structures in the target. The average size of target segments following hybridization is usually larger than the size of probe on the chip.

In one example of the present invention, PCR reactions were performed in a 25-μl volume containing 10 ng of genomic DNA or 1 ng of purified BAC DNA, 1 mM of each primer, 2.5 units of AmpliTaq Gold (Perkin-Elmer), 0.25 mM deoxynucleotide triphosphates (dNTPs), 10 mM tris-HCl (pH 8.3), and 50 mM KCl, and 1.25 mM MgCl₂. Thermocycling was performed on a 9600 or 9700 automated thermal cycler (Perkin-Elmer), with initial denaturation at 95° C. for 10 min, followed by one of two cycling conditions based on the melting temperature of the primers: either 10 cycles of [94° C. 30 sec, 58° C. 30 sec, 72° C. 30 sec] followed by 30 cycles of [94° C. 30 sec, 55° C. 30 sec, 72° C. 30 sec] or 10 cycles of [94° C. 30 sec, 55° C. 30 sec, 72° C. 30 sec] followed by 30 cycles of [94° C. 30 sec 52° C. 30 sec, 72° C. 30 sec]. A final extension reaction was carried out at 72° C. for 5 min. The amplified DNA was then purified using the Qiagen Large-Construct Kit (Qiagen), fragmented with deoxyribonuclease (DNase) 1 (Boehringer Manneheim) and labeled with biotin with terminal deoxynucleotidyl transferase (TdT, GibcoBRL Life Technology). Fragmentation was performed in a 74-μl volume with 0.2 unit of DNase 1, 10 mM tris-acetate (pH 7.5), 10 mM magnesium acetate, and 50 mM potassium acetate at 37° C. for 10 min, after which the reaction was stopped by heat inactivation at 99° C. for 10 min. The terminal transferase reaction was performed by adding 50 units of TdT and 12.5 μM biotin-N6-ddATP (Dupont NEN) to the preceding reaction mix, incubating at 37° C. for 90 min, and then heat-inactivating at 99° C. for 10 min.

V. Array Design

The methods described above typically utilize an array for basecalling of the sequence from the second organism. As such, a substrate having immobilized thereon a plurality of detection probes is provided. Preferably, each detection probe is at a known location. The plurality of probes can be at any density that is useful to practice the invention. Substrates with a plurality of probes are known in the art. In specific preferred embodiments the density is at least 100 probes/cm²; or is at least 1,000 probes/cm²; or is at least 10,000 probes/cm².

In practicing the invention one skilled in the art knows how to determine the best length of probe to further hybridization. In preferred embodiments of the present invention the probes are at least 18 bases long or are at least 20 bases long or are at least 25 bases long.

The detection probes are derived from a first nucleic acid sequence, which can be from any organism, provided that the sequence of the nucleic acid is known. In one application of the present invention, the first nucleic acid sequence is derived from a human. In one preferred embodiment, genomic DNA is used.

Preferably, at least one of said detection probes is complementary to a known human nucleic acid sequence and at least one of the detection probes is non-complementary to a known human nucleic acid sequence. Preferably, the probes that are non-complementary are designed to be one-base mismatch non-complementary to genomic sequence derived from a human (the reference sequence).

Methods for designing, selecting and making probe sets are described in, for example, WO 95/11995, WO 92/10092, or U.S. Pat. Nos. 5,143,854; 5,384,261; 5,405,783; 5,412,087; 5,424,186; 5,445,934; 5,744,305; 5,800,992; 6,040,138; 6,040,193, all of which are incorporated herein by reference for all purposes. One with skill in the art would appreciate that the detection arrays of the present invention are not limited to one particular manufacturing method. For example, oligonucleotide probes may be pre-synthesized and deposited on a substrate. Detection, as used herein, refers to processes including identifying base composition and sequence of a target sequence based upon the known sequence of a reference nucleic acid. The detection probe arrays or chips are designed using this reference sequence, typically the genomic sequence of a first organism.

One strategy for array design provides an array that is subdivided into sets of four probes (oligonucleotides of differing sequence), although in some situations, more or less probes per set may be appropriate. In a typical embodiment, one probe in each probe set comprises a plurality of bases exhibiting perfect complementarity with a selected reference sequence (i.e., the genomic sequence of a first species). In this probe of the set, complementarity with the reference sequence exists throughout the length of the probe. For the other three probes in the set, complementarity with the reference sequence exists throughout the length of the probe except for an interrogation position, which typically consists of one nucleotide base at or near the center of probe. For example, for an A nucleotide in the reference sequence, the corresponding probe with perfect complementarity from the probe set has its interrogation position occupied by a T, the correct complementary base. The other probes from the set have their respective interrogation positions occupied by A, C, or G—a different nucleotide in each probe. Thus, there are four probes corresponding to each nucleotide of interest in the reference sequence. Alternative embodiments exist, however, and the present invention should not be limited to arrays with four probes per probe set. A five-probe per set embodiment is described infra.

The probes can be oligodeoxyribonucleotides or oligoribonucleotides, or any modified forms of these polymers that are capable of hybridizing with a target nucleic sequence by complementary base-pairing. Complementary base pairing means sequence-specific base pairing which includes e.g., Watson-Crick base pairing as well as other forms of base pairing such as Hoogsteen base pairing. Modified forms include 2′-O-methyl oligoribonucleotides and so-called PNAs, in which oligodeoxyribonucleotides are linked via peptide bonds rather than phosphodiester bonds. The probes can be attached by any linkage to a support (e.g., 3′, 5′ or via the base). Attachment at the 3′ end of the probe is usual as this orientation is compatible with the preferred chemistry for solid phase synthesis of oligonucleotides.

For simplicity, the sets are usually arranged in order of the reference sequence in a horizontal row across the array, though other embodiments are used. A horizontal row contains a series of overlapping probes with the same base at the interrogation position. These overlapping probes span the selected reference sequence. Each set of four probes usually differs from the previous set of four probes by the omission of a base at one end and the inclusion of an additional base at the other end. However, this orderly progression of probes may be interrupted by the inclusion of control probes or the omission of certain probes in rows or columns of the array. In addition, probes may be placed so as to orient the array, or gauge the background or non-specific binding of the sample to the array. One of skill in the art would appreciate that the probes may not be necessarily arranged in such an order as described above, but could be in any order as long as the sequence of a probe can be correlated to location on the array.

The sets of probes are usually laid down in horizontal rows such that all probes having an interrogation position occupied by an A form an “A row” in the vertical direction, all probes having an interrogation position occupied by a C form a “C row”, all probes having an interrogation position occupied by a G form a “G row”, and all probes having an interrogation position occupied by a T (or U) form a T row (or a U row).

In most arrays, all probes are the same length. Optimum probe length may vary depending on, among other things, the GC content of a particular region of the target DNA sequence, secondary structure, synthesis efficiency and cross-hybridization. The appropriate size of probes at different regions of the target sequence can be determined by comparing the readability of different sized probes in different regions of a target.

In preferred embodiments of the present invention, the arrays are designed to have sets of probes complementary to both strands of the reference sequence (coding or non-coding). Independent analysis of coding and non-coding strands provides largely redundant information; however, the regions of ambiguity in reading the coding strand are not always the same as those in reading the non-coding strand. Thus, combination of the information from coding and non-coding strands increases the overall accuracy of the sequence data.

VI. Hybridization Assay

A. Hybridization Step

Hybridization assays on a substrate-bound oligonucleotide arrays involve a hybridization step and a detection step. In the hybridization step, a hybridization mixture containing the target and, typically, an isostabilizing agent, denaturing agent or renaturation accelerant, is brought into contact with the probes of the array and incubated at a temperature and for a time appropriate to allow hybridization between the target and any complementary probes. Usually, unbound target molecules are then removed from the array by washing with a wash mixture that does not contain the target, leaving only bound target molecules.

The hybridization mixture includes the target nucleic acid molecule and hybridization optimizing agents in an appropriate solution (buffer). The target nucleic acid is present in the mixture at a concentration between about 0.005 nM target per ml hybridization mixture and about 50 nM target per ml hybridization mixture. The hybridization mixture is placed in contact with the array and incubated. Generally, incubation will be at temperatures normally used for hybridization of nucleic acids, for example, between about 25° C. and 65° C. For probes longer than 14 nucleotides, a temperature range of 37° C. and 45° C. is preferred. Incubation time varies, but can be as short as 30 minutes and as long as 12 hours or more. After incubation with the hybridization mixture, the array is usually washed with buffer. Examples of general hybridization conditions may be found in many sources, including: Sambrook, et al., Molecular Cloning: A Laboratory Manual (1989), 2d Ed., Cold Spring Harbor, N.Y.; Berger and Kimmel, “Guide to Molecular Cloning Techniques”, Methods in Enzymology (1987), Vol. 52, Academic Press, Inc.; Young and Davis, Proc. Natl. Acad. Sci. (USA) 80:1194 (1983). Hybridization conditions specific for oligonucleotide arrays can be found in product literature from Affymetrix, Inc. (Santa Clara, Calif.) and U.S. Pat. No. 6,045,996 to Cronin et al. In one example of the present invention, DNA labeling and hybridization to arrays was performed as described in D. G. Wang et al., Science 280:1077 (1998), with minor modifications. The labeled DNA sample was denatured in hybridization buffer [3M tetramethylammonium chloride, 10 mM tris-HCl (pH 7.8), 0.01% Triton X-100, herring sperm DNA (100 μg/ml), and 50 pM control oliogomer] at 99° C. for 5 min and hybridized to an oligonucleotide array overnight at 40° C. on a rotisserie at 40 rpm. All washes and staining were performed at room temperature. Oligonucleotide arrays were washed twice with 1×MES buffer [0.1 M 2-[N-Morpoline]ethanesulfonic acid (pH 6.7), 1 M NaCl, and 0.01% Triton X-100], and stained with staining solution [streptavidin R-phycoerythrin (20 μg/ml) (Molecular Probes) and acetylated bovine serum albumin (BSA) (1 mg/ml) in 2×MES] for 20 min on a rotisserie at 40 rpm. Following two washes with 1×MES, chips were incubated with antibody solution [biotinylated anti-streptavidin antibody (10 μg/ml) and BSA (1 mg/ml) in 2×MES] for 20 min on a rotisserie at 40 rpm. After two washes with 1×MES, arrays were stained again with staining solution for 20 min. The oligonucleotide arrays were washed 6 times with 6×SSPET [0.9 M NaCl, 60 mM NaH₂PO₄, 6 mM EDTA (pH 7.4), 0.01% Triton X-100] at 35° C. on a fluidics workstation (Affymetrix).

Determining a signal generated from a detectable label on an array requires an oligonucleotide array or chip reader. The nature of the oligonucleotide array reader depends upon the particular type of label attached to the target molecules. A typical reader employs a system where the light source is placed above the array to be scanned and a photodiode detector is below the array. A preferred reader replaces the photodiode with a CCD camera and imaging optics to allow rapid imaging of the array. In one example of the present invention, hybridization of target DNA to the array was detected by using a custom confocal scanner with a resolution of 110 pixels per feature (pixel size of 2.27 μM) and 560-nm filter.

B. Detection Step

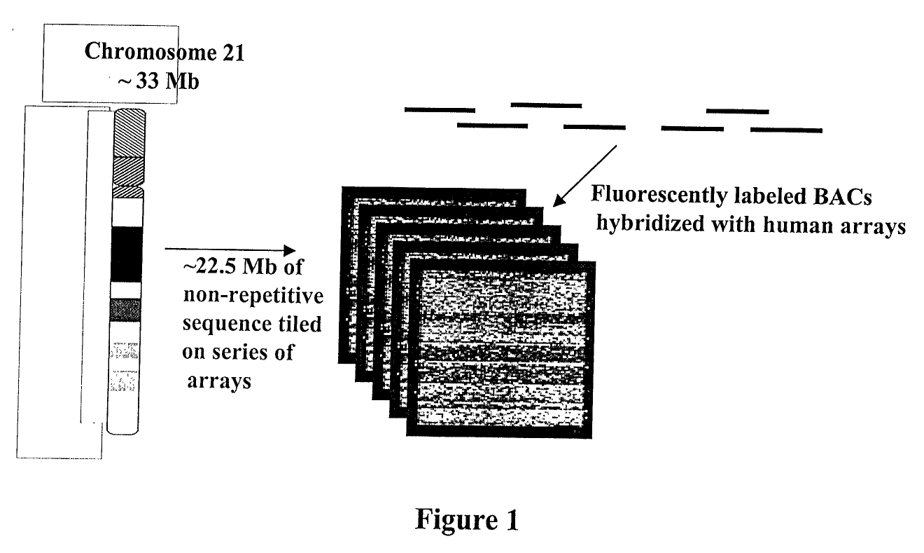

The arrays are read by comparing the intensities of labeled target nucleotides (amplified genomic DNA from the second species) that are bound to the probes (oligonucleotides engineered to be complementary to the sequence of genomic DNA of a first species) on an array after hybridization (in general, see FIGS. 1-3). Specifically, a comparison is performed between each probe (e.g., probes differing in their interrogation position by an A, C, G and T) of each probe set. For a particular probe set, the probe position showing the greatest hybridization signal is called as the nucleotide present at the position in the target sequence corresponding to the interrogation position in the probes. Clearly, of the four probes in a set, only one can exhibit, for example, a perfect match to the target sequence whereas the other probes of the set exhibit at least a one base pair mismatch. However, in some regions of the target sequence, the distinction between a perfect match and a one-base mismatch is less clear, or, frequently, there may be more than one mismatched base, in which case one probe will have, instead of perfect complementarity, one base greater complementarity than the other probes of the set. The probe exhibiting the best match usually produces substantially greater hybridization signal than the other three probes in the column and is thereby easily identified. In one embodiment of the present invention, the probe with the best hybridization signal is called as the sequence nucleotide. In other embodiments of the present invention, a call ratio is established to define the ratio of signal from the best hybridizing probes to the second best hybridizing probe that must be exceeded for a particular target position to be read from the probes. A high call ratio ensures that few if any errors are made in calling target nucleotides, but can result in some nucleotides being scored as ambiguous, which could in fact be accurately read. A lower call ratio results in fewer ambiguous calls, but can result in more erroneous calls. It has been found that at a call ratio of 1.2, virtually all calls are accurate.

For target sequences showing a high degree of divergence from the reference strain or incorporating several closely spaced mutations from the reference strain, a single set of probes (i.e., designed with respect to a single reference sequence) will not always allow accurate sequence to be called. Target sequence bearing insertions will may exhibit short regions including and proximal to the insertion that usually cannot be read. The presence of short regions of difficult-to-read target because of closely spaced mutations, insertions or deletions, does not prevent determination of the remaining sequence of the target as different regions of a target sequence are determined independently.

When the arrays comprise four-probe sets, and the probe sets are laid down in columns to form rows—an A row, a C row, a G row and a T or U row—the probe having a segment exhibiting perfect complementarity to a reference sequence varies between the columns from one row to another. This does not present any significant difficulty in computer analysis of the data from the array. However, visual inspection of the hybridization pattern of the array is sometimes facilitated by provision of an extra probe (a fifth probe in each set), which exhibits perfect complementarity to the reference sequence. This fifth probe is identical to one of the other probes of the set. The extra probes may be placed to form a row (designated the wildtype row) and would hybridize to a target sequence at all nucleotide positions except those in which deviations from the reference sequence occurs. The hybridization pattern of the wildtype row thereby provides a simple visual indication of sequence similarity and dissimilarity.

VII. Methods for Analysis

According to the present invention, various statistic parameters based on a comparison of the nucleic acid sequence of a first organism (the reference sequence) and the nucleic acid of a second organism are computed. These may include conformance for all windows of a given size and overlap. In other words, for a 30 base pair window with a 10-base pair overlap, conformance is computed for base pairs 1-30, 21-50, 41-70, and so on, as the percent of probes matching the reference sequence (of the 60 probes—30 for the Watson strand, 30 for the Crick strand). The distance of each window from the nearest known repeat is also computed, masking the repeat regions on the reference sequence. In addition, the maximum frequency of any base in the reference sequence corresponding to each window is computed. Finally, the maximum frequency of any base within a sub-window of a given length (e.g., 15 base pairs) within the reference sequence is computed for each window.

After these statistics are computed, windows are classified as potentially conserved for those which (a) conformance is at some percent, (b) nearest repeat is at some distance, (c) maximum single-base frequency is less than some percent, and (d) maximum single-base frequency for any 15-base pair sub-window is less than some percent. Then, for all potentially-conserved windows within so many base pairs of another potentially-conserved window, the windows between them are classified as potentially conserved. Finally, from the collection of potentially-conserved windows, the potentially-conserved contiguous regions are computed.

In one embodiment, an identity index, such as percentage of similarity, in a plurality of sub-regions of the nucleic acid sequences are calculated. Sub-regions are overlapping, moving “windows” of base pairs of sequence across the longer sequence. The size of the windows may be adjusted or may be varied, depending on the relatedness of the organisms being compared. Preferably, the window is at least 20 base pairs in length and can be up to 150 base pairs in length, with overlapping bases of 5 to 75 bases for each window. In one embodiment of the present invention, windows of 30 base pairs with 10 base pairs overlap between each window were used. Determining whether the sequence identity between the first and second sequences is high enough to indicate a functional region requires setting a threshold or significance value for sequence identity (percentage of bases that are identical between the two organisms within said sub-region). In practice, a useful selection of this threshold can be done fairly easily and is done commonly. Significance values will differ depending on the relatedness between the organisms, and will be higher the more closely related the organisms.

More specifically, the sequence of a reference sequence with n bases (i.e., the sequence of nucleic acids from the first organism) is provided:

S=(s₁, s₂, . . . , sₙ)

where

sᵢ∈{A,C,G,T}, i=1, . . . ,n

Preferably, repeats of the reference sequence have been masked to give:

M=(m₁, m₂, . . . , mₙ)

with

mᵢ∈{A,C,G,T,N}, i=1, . . . ,n

where

$${m}_{i} = \{ \begin{matrix}N , \\ {s}_{i} ,\end{matrix} \begin{matrix}if {s}_{i} is in a repeat region \\ otherwise\end{matrix}$$

Basecalls for the forward strand from a nucleic acid sequence of a second organism are generated:

F=(f₁, f₂, . . . , fₙ)

where

fᵢ∈{A,C,G,T}, i=1, . . . ,n

Likewise, basecalls for the reverse strand from a nucleic acid sequence of a second organism are generated:

R=(r₁, r₂, . . . , rₙ)

where

rᵢ∈{A,C,G,T}, i=1, . . . ,n

The overlapping, “moving windows” of fixed length for the reference sequence, masked sequence, and called sequences are defined, where

h=length in bases of each window

v=minimum amount of overlap between any two adjacent windows

u=h−v=offset from the start of all (except possibly the next to last window) to the start of the next

The set of windows generated by these parameters for the reference sequence S are then called:

W_{S}=(w_{S,1}, w_{S,2}, . . . , w_{S,k})

so that

w_{S,1}=(s₁, s₂, . . . , sₕ)

w_{S,2}=(sᵤ₊₁, sᵤ₊₂, . . . , sᵤ₊ₕ)

. . .

w_{S,i}=(s₍ᵢ₋₁₎ᵤ₊₁, . . . , s₍ᵢ₋₁₎ᵤ₊ₕ), i<k

while the last interval, which is required to have length h and terminate with the final base in the sequence is

w_{S,k}=(sₙ₋ₕ₊₁, sₙ₋ₕ₊₂, . . . , sₙ)

Note that this last window may have an overlap of more than v bases with the previous window. Also, note that

$$k = ⌈ \frac{n - v}{u} ⌉ , n > v$$

Similar windows for the masked sequence (W_{M}), the basecalls for the forward strand (W_{F}), and the basecalls for the reverse strand (W_{R}) are defined.

Conserved regions are determined. This begins by computing a number of statistics for each window. Specifically, conformance (the measure of how well the experiment matches the reference sequence) is determined:

$${c}_{i} = \frac{1}{2 h} {\sum }_{j = 1}^{h} match ( {w}_{S , i , j} , {w}_{F , i , j} ) + match ( {w}_{S , i , j} , {w}_{R , i , j} )$$

where

w_{S,i,j}=jth base in the ith window of the reference sequence,

w_{F,i,j}=jth base in the ith window of the basecalls of the forward sequence, and

w_{R,i,j}=jth base in the ith window of the basecalls of the reverse sequence

and

$$match ( x , y ) = \{ \begin{matrix}1 , \\ 0 ,\end{matrix} \begin{matrix}if x = y \\ otherwise\end{matrix}$$

In other words, cᵢ is the proportion of basecalls that match the reference sequence over both the forward and reverse strands for the ith window.

The maximum single-base frequency, a measure of local complexity of the reference sequence, is determined:

bᵢ=max(n_{A,i}, n_{C,i}, n_{G,i}, n_{T,i})

where

n_{X,i}=frequency of base X in window w_{S,i}

The maximum single-base frequency over a subwindow (another measure of local complexity) is determined. Let h*<h be a fixed number of bases, fewer than the window size. Then let

n_{X,i,j}=frequency of base X in subwindow w_{S,i,j}

where subwindow j of window i starts at the jth base

w_{S,i,J}=(sₚ_{ᵢ}_{+J-1}, sₚ_{ᵢ}_{+j+h*-1})

for j=1, 2, . . . , h−h*, where pᵢ is the starting base of the ith window. Note that, except (possibly) for the last window,

pᵢ=(i−1)u+1

and, for the last window

pₖ=n−h+1

Finally, define b₂ to be the maximum of the n_{X,i,j}.

The distance from nearest repeat window is determined. A “repeat window” is a window in which at least one base pair overlaps a repeat region in the masked sequence M. Again, let pi be the starting base of the ith window. Then

$${d}_{i} = {min}_{j} \{ \vert {p}_{i} - {p}_{j} + 1 \vert , where {p}_{j} is the start of a repeat window$$

Note that dᵢ is zero for a repeat window.

After computing statistics for the k windows, individual windows are marked as conserved if each of the four statistics has acceptable values. In particular, window i is considered conserved if and only if:

cᵢ≧T_{c}

where T_{c} is the conformance threshold, and

bᵢ<T_{b}

where T_{b} is the maximum single-base pair frequency threshold, and

b₂ᵢ<T_{b2}

where T_{b2} is the maximum single-base pair frequency over subwindows threshold, and finally

dᵢ>T_{d}

where T_{d} is the threshold for minimum distance from a repeat window.

Once windows have been determined conserved by the various statistics, additional windows in small gaps between conserved windows are also declared conserved. If window i is conserved, and window i+j−1 is conserved, where j<T_{g}, and if windows i+1, i+2, . . . , i+j−2 are not conserved, then the windows between i and i+j−1 are also marked as conserved.

Finally, conserved regions are defined as subsequences of conserved windows. Call the set of conserved regions

CR=(cr₁, cr₂, . . . , crₘ)

where

crᵢ=(xᵢ, yᵢ)

indicating that the ith conserved region corresponds to bases xᵢ to yᵢ of the sequence S. If window i₁ is the first conserved window, and windows i₁+1, i₁+2, . . . , j₁ are also conserved, while window j₁+1 is not conserved, then

(x₁, y₁)=(pᵢ_{₁}, qⱼ_{₁})

where pᵢ is the start of the ith window as before, and qⱼ is the ending base of the jth window. Similarly, if i₂ is the first conserved window after j₁ (hence, i₂>j₁+1), and windows i₂+1, i₂+2, . . . , j₂ are all conserved, but window j₂+1 is not (or, possibly j₂ is the last window), then

(x₂, y₂)=(pᵢ_{₂}, qⱼ_{₂})

The other conserved regions are defined similarly, up to the mth, where jₘ is the last conserved window, and windows iₘ, iₘ+1, jₘ−1 are all conserved, but window iₘ−1 is not.

VIII. Applications

A. Use in Identification of Conserved Sequences

Sequences were classified as conserved on the basis of high conformance. Conformance was computed as the percent of perfect-match probes that had greater fluorescent intensity than the corresponding mismatch probes over sequences of 30 basepairs. That is, if the 25-mer probe complementary to the reference sequence (as opposed to one of the three probes with a mismatch to the reference sequence at the 13^{th} nucleotide) had the highest intensity of the four probes, then 1 was added to the total conformance for the interval. Therefore, if 8 of 30 bases had the perfect-match probe having the highest intensity on the Watson strand, and 7 perfect-match probes had the highest intensity on the Crick strand, the conformance would be (8+7)/(30+30)=25%.

Conformance was computed for base pairs 1-30, 21-50, 41-70, and so on for each sequence fragment tiled on the arrays. Interspersed repeats were not tiled on the arrays; therefore, sequence fragments of differing lengths were present. For a sequence fragment of 100 bp, conformance would be computed for five overlapping intervals, with the fifth interval being base pairs 71-100. This was to maintain an interval width of exactly 30 bp with a minimum overlap of 10 bp, such that every base appeared in at least one interval. Based on examination of known false positives and verified conserved sequences, criteria were developed to classify a 30-bp interval as conserved. An interval was classified conserved if:

(1) conformance was ≧60%;

(2) an interspersed repeat did not exist in an overlapping interval (within 20 basepairs),

(3) the maximum frequency of any one base in the reference sequence was <15, and

(4) the maximum frequency of any one base was <10 in any 15 consecutive base pairs.

Criteria (2), (3), and (4) eliminated intervals in which high levels of hybridization occurred solely because of the repetitive or low-complexity (e.g., a sequence of “ATATAT . . . AT”) nature of the reference sequence. After determining which 30-bp intervals were conserved, the conserved elements were derived from merging overlapping conserved intervals. If, for example, the intervals containing base pairs 131-160, 151-180, and 171-200 were conserved, but not the intervals before and after them, then this would constitute a single conserved element from base pairs 131-200, with length 70 bp.

For example, in comparing sequence between a mouse and a human, it was determined that windows of 30 base pairs with an overlap of 10 base pairs that had conformance of 60% or higher showed strong similarity between human and mouse, except in cases where the window was close to or within a repeat or a region of low complexity. Further, it was determined that windows that were within 20 base pairs of a repeat sometimes showed spuriously high conformance. Likewise, analysis of windows with high conformance led to the decision that windows in which the reference sequence had either (a) 50% or more of a single base (either A, G, T, or C) or (b) 67% or more of a single base within any sub-window of 15 base pairs within the 30-base pair window (i.e., 10 or more out of 15), would sometimes show high conformance. Such sequences of low local complexity were considered not of interest, and were therefore not classified as potentially conserved. Further inspection of sequence similarities led to the conclusion that nearby windows with high conformance were likely to be parts of the same potentially-conserved element. For example, there were clear cases where an exon was conserved and most, but not all, windows covering that exon showed high conformance. Thus, it was determined that regions of 120 base pairs or less between potentially conserved-windows would also be declared as potentially conserved.

B. Use in the Identification of Conserved Sequences in the Human Genome

The methods of the invention can be used to identify conserved sequences in the human genome. Conserved elements are merged when the distance (gap size parameter) between the elements is less than or equal to 15; and the elements obey one of the following rules:

1. The two elements are unique to one species;

2. The two elements overlap in all species; or

3. The two elements overlap the exact same subset of species.

C. Use in the Identification of Expressed Elements

The sequence tiled on the high-density arrays was analyzed in blocks of 30 base pairs, with each block overlapping the next by 10 base pairs. Each block is determined to be either expressed or not based on two block-averaged measures—the average conformance and the average intensity ratio. The average conformance was evaluated as the fraction of matches (i.e. bases for which the probe corresponding to the reference sequence was brighter than the three probes corresponding to mismatches) over the thirty bases in the block, averaged over the two strands. The intensity ratio at each base was computed as the ratio of the intensity at the brightest probe to that at the probe next in intensity.

The background signal for this analysis was determined from an experiment in which RNA selected using long-range PCR products corresponding to ˜4.2 Mb of Chromosome 21 sequence were hybridized with a microarray on which 2.9 Mb of non-overlapping Chromosome 21 sequence was tiled. Histograms were accumulated for the distributions of average conformance and intensity ratio for this background experiment. Based on these, stringent criteria for high specificity (and correspondingly lower sensitivity) were developed for the identification of expression from high-density arrays: blocks with an average conformance of at least 70% and an average intensity ratio of at least 1.2 were identified as being expressed. Overlapping adjacent blocks of expressed sequence identified in this manner were combined into elements for further analysis. In analyzing the signal and background as well as in identifying expressed elements, blocks were excluded from the analysis if the frequency of any one base in the reference sequence was greater than or equal to 10; or if the frequency of any one base exceeded 10 in any consecutive 15 base pairs within a block. No blocks in the background experiment fulfilled these criteria for identifying expression. Thus, the false positive rate indicated for these criteria is less than 7×10⁻⁶.

D. Use in the Identification of Expressed Elements or in the Identification of Deletions

Tiled sequence is divided into blocks of 30 bp, overlapping by 20 bp. A block is identified as part of a potential deletion if (i) the conformance within the block is no more than 45%; and the amplicon containing the block has a conformance of at least 75%. Overlapping blocks of low conformance are merged together into single elements.

For example, the methods of the invention have resulted in the detection of a 250 base pair deletion on chromosome 21 in humans. Six of 20 copies of chromosome 21 that have been examined contain the deletion.

VIII. Apparatus

Further, the invention also provides computational methods and computer software products are provided for sequence comparison between organisms. Such computational methods and computer software products may involve computer software that receives a plurality of hybridization signal intensities from a hybridized array from a detector. The hybridization signal intensities reflect the amount of hybridization of the nucleic acid sample (derived from the second organism) to the detection probes (derived from the sequence of the first organism). Further, such computational methods and computer software may also produce and include, respectively, software modules that identify bases of the sequence of the second organism according to the hybridization intensities. In some applications, the computational methods and computer software produce and include, respectively, functionality that allows an operator to select window size, used to calculate the identity ratio, and a threshold value. When the identity ratio of a region is above the threshold value, a putative functional region of the genome is identified.

Generally, embodiments of the present invention employ various processes involving data stored in or transferred through one or more computer systems. Embodiments of the present invention also relate to an apparatus for performing these operations. This apparatus may be specially constructed for the required purposes, or it may be a general-purpose computer selectively activated or reconfigured by a computer program and/or data structure stored in the computer. The processes presented herein are not inherently related to any particular computer or other apparatus. In particular, various general-purpose machines may be used with programs written in accordance with the teachings herein, or it may be more convenient to construct a more specialized apparatus to perform the required method steps. A particular structure for a variety of these machines will appear from the description given below.

In addition, embodiments of the present invention relate to computer readable media or computer program products that include program instructions and/or data (including data structures) for performing various computer-implemented operations. Examples of computer-readable media include, but are not limited to, magnetic media such as hard disks, floppy disks, and magnetic tape; optical media such as CD-ROM disks; magneto-optical media; semiconductor memory devices, and hardware devices that are specially configured to store and perform program instructions, such as read-only memory devices (ROM) and random access memory (RAM). The data and program instructions of this invention may also be embodied on a carrier wave or other transport medium. Examples of program instructions include both machine code, such as produced by a compiler, and files containing higher level code that may be executed by the computer using an interpreter.

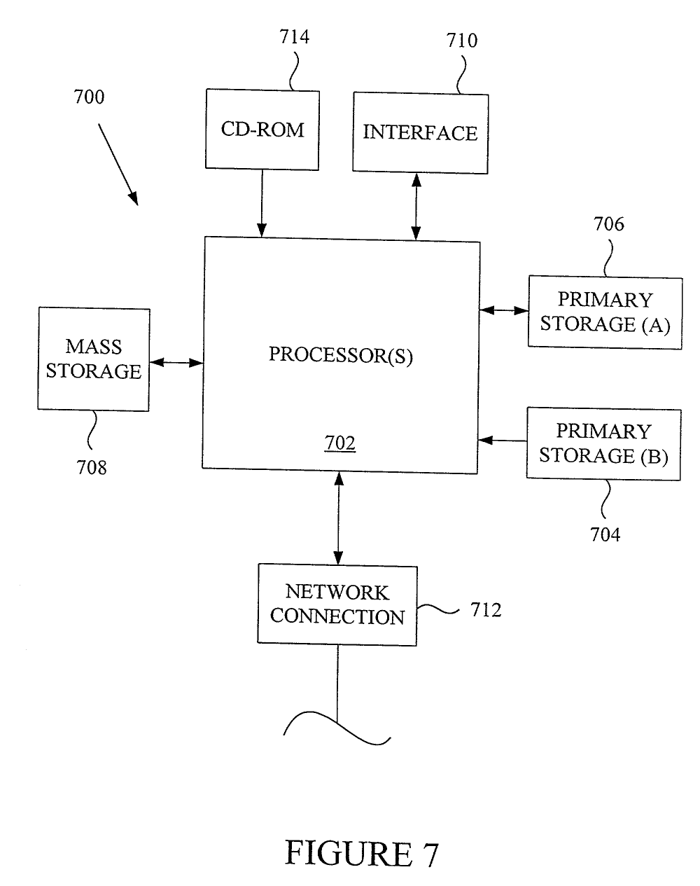

FIG. 7 illustrates a typical computer system that, when appropriately configured or designed, can serve as an image analysis apparatus of this invention. The computer system 700 includes any number of processors 702 (also referred to as central processing units, or CPUs) that are coupled to storage devices including primary storage 706 (typically a random access memory, or RAM), primary storage 704 (typically a read only memory, or ROM). CPU 702 may be of various types including microcontrollers and microprocessors such as programmable devices (e.g., CPLDs and FPGAs) and unprogrammable devices such as gate array ASICs or general purpose microprocessors. As is well known in the art, primary storage 704 acts to transfer data and instructions uni-directionally to the CPU and primary storage 706 is used typically to transfer data and instructions in a bi-directional manner. Both of these primary storage devices may include any suitable computer-readable media such as those described above. A mass storage device 708 is also coupled bi-directionally to CPU 702 and provides additional data storage capacity and may include any of the computer-readable media described above. Mass storage device 708 may be used to store programs, data and the like and is typically a secondary storage medium such as a hard disk. It will be appreciated that the information retained within the mass storage device 708, may, in appropriate cases, be incorporated in standard fashion as part of primary storage 706 as virtual memory. A specific mass storage device such as a CD-ROM 714 may also pass data uni-directionally to the CPU.

CPU 702 is also coupled to an interface 710 that connects to one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 702 optionally may be coupled to an external device such as a database or a computer or telecommunications network using an external connection as shown generally at 712. With such a connection, it is contemplated that the CPU might receive information from the network, or might output information to the network in the course of performing the method steps described herein.

In one embodiment, the computer system 700 is directly coupled to a hybridization detector or scanner. Data from the detector are provided via interface 712 for analysis by system 700. Alternatively, the data or hybridization signal intensities processed by system 700 are provided from a data storage source such as a database or other repository. Again, the data are provided via interface 712. Once in the computer system 700, a memory device such as primary storage 706 or mass storage 708 buffers or stores, at least temporarily, the data or hybridization intensities. With this data, the image analysis apparatus 700 can perform various analysis operations such as calculating intensities indices and the like. To this end, the processor may perform various operations on the stored images or data.

The invention thus also provides for an apparatus for identifying evolutionarily conserved sequences. This apparatus comprises a scanner for scanning hybridization intensities; a first memory region for storing data said hybridization intensities; a second memory region for storing process steps; and a processor for executing the process steps stored in said second memory region; wherein said second memory region includes process steps to (a) receive a plurality of hybridization intensities wherein each of said intensities reflects the hybridization of one of a plurality of probes from a first nucleic acid sequence from a first organism to a sample nucleic acid from a second organism, wherein said probes are complementary and non-complementary to a known nucleic acid sequence from said first organism, wherein said probes are arrayed on a substrate and wherein each detection probe is at a known location on said substrate, (b) identify bases of said plurality of probes according to said hybridization intensities, and (c) calculate various statistic parameters between said first nucleic acid sequence from said first organism and said sample nucleic acid from said second organism. The apparatus optionally further comprises a database including said parameters and hybridization intensities.

Another embodiment of the present invention is drawn to a computer program product comprising a machine readable medium on which is provided program instructions for identifying evolutionarily conserved and/or divergent sequences. The instructions comprises code for receiving a plurality of hybridization intensities wherein each of the intensities reflects the hybridization of one of a plurality of probes from a first nucleic acid sequence from a first organism to a sample nucleic acid from a second organism, wherein the probes are complementary and non-complementary to a known nucleic acid sequence from the first organism, wherein the probes are arrayed on a substrate and wherein each detection probe is at a known location on the substrate; code for identifying bases of the plurality of probes according to the hybridization intensities; and code for calculating various statistical parameters between the first nucleic acid sequence from the first organism and the sample nucleic acid from the second organism. In a further embodiment, the computer program product further comprises code for storing and retrieving hybridization intensities and various statistical parameters.

The invention also provides for a computing device comprising a memory device configured to store at least temporarily program instructions for identifying evolutionarily conserved and/or divergent sequences, the instructions comprising: code for receiving a plurality of hybridization intensities wherein each of the intensities reflects the hybridization of one of a plurality of probes from a first nucleic acid sequence from a first organism to a sample nucleic acid from a second organism, wherein the probes are complementary and non-complementary to a known nucleic acid sequence from the first organism, wherein the probes are arrayed on a substrate and wherein each detection probe is at a known location on the substrate; code for identifying bases of the plurality of probes according to the hybridization intensities; and code for calculating various statistical parameters between the first nucleic acid sequence from the first organism and the sample nucleic acid from the second organism. In a further embodiment, the computing device further comprises code for storing and retrieving hybridization intensities and various statistic parameters.

IX. Applications

In one aspect, methods are provided for determining sequence similarity between nucleic acids from a first organism and nucleic acids from a second, different organism without knowing a nucleic acid sequence from the second, different organism. In one application of the present invention, the fist nucleic acid is derived from a human and the second nucleic acid is derived from another animal species. According to this aspect, the second organism diverged evolutionarily from the first organism between about 60 million years ago and about 120 million years ago.

In a specific embodiment of the invention, there is a method for determining sequence similarity between nucleic acids from a first organism and a second organism, comprising: providing a substrate having a plurality of detection probes, wherein each detection probe is at a known location, and wherein at least one of said detection probes is complementary to a known nucleic acid sequence from said first organism and at least one of said detection probes is non-complementary to a known nucleic acid sequence from said first organism; contacting at least one sample nucleic acid from said second organism with said substrate under conditions wherein when said at least one sample nucleic acid is substantially complementary to a detection probe said at least one sample nucleic acid will preferentially hybridize to a detection probe to which it is most complementary resulting in at least one hybridized detection probe; determining a location of said at least one hybridized detection probe; and identifying sequences of said at least one hybridized detection probe by referring to the location of said at least one hybridized detection probe; wherein when said sequence of said at least one hybridized detection probe is the same as a sequence complementary to said known nucleic acid sequence from said first organism, there is sequence similarity between nucleic acids from said first organism and said second organism.

In a second aspect, methods are provided to screening for functional regions of a first genome from a first organism, by comparing the genomic sequence from the first organism with the genomic sequence of a second organism without knowing a nucleic acid sequence from the second organism. The method involves determining which bases from the nucleic acid from the second species are identical to the bases from the nucleic acid of the first species. Regions where the number of identical bases is above a pre-determined threshold value are regions of putative functional significance in the first species.

Another specific embodiment of the invention includes a method for screening for functional sequences in a genome of a first organism, comprising: providing a substrate having a plurality of detection probes, wherein each detection probe is at a known location, and wherein at least one of said detection probes is complementary to a known nucleic acid sequence in the genome from said first organism and at least one of said detection probes is non-complementary to a known nucleic acid sequence in the genome from said first organism; contacting at least one sample nucleic acid from a second organism with said substrate, where said second organism diverged evolutionarily from said first organism between about 60 million years ago and about 120 million years ago, and where said contacting is performed under conditions wherein when said at least one sample nucleic acid is substantially complementary to a detection probe said at least one sample nucleic acid will preferentially hybidrize to a detection probe to which it is most complementary, resulting in at least one hybridized detection probe; determining a location of said at least one hybridized detection probe; and identifying sequences of said at least one hybridized detection probe by referring to the location of said at least one hybridized detection probe; wherein when said sequence of said at least one hybridized detection probe is the same as a sequence complementary to said known nucleic acid sequence from said first organism, there is sequence similarity between nucleic acids from said first organism and said second organism, and regions in said nucleic acids of said first organism where there is sequence similarity with said nucleic acids from said second organism are candidate functional regions in said nucleic acids of said first organism.

In a third aspect, the invention further provides enhanced methods for analysis of functional regions of a genome. Such methods entail determining regions of a genome that are conserved between a plurality of organisms. Additional specific embodiments of the invention include a method for screening for functional sequences in nucleic acids of a first organism, comprising: providing a first substrate having a plurality of detection probes, wherein each detection probe is at a known location, and wherein at least one of said detection probes is complementary to a known nucleic acid sequence from said first organism and at least one of said detection probes is non-complementary to a known nucleic acid sequence from said first organism; contacting at least one sample nucleic acid from a second organism with said first substrate under conditions wherein when said at least one sample nucleic acid is substantially complementary to a detection probe said at least one sample nucleic acid will preferentially hybridize to a detection probe to which it is most complementary, resulting in at least one hybridized detection probe; determining a location of said at least one hybridized detection probe; and identifying sequences of said at least one hybridized detection probe by referring to the location of said at least one hybridized detection probe; wherein when said sequences of said at least one hybridized detection probe is the same as a sequence complementary to said known nucleic acid sequence from said first organism, there is sequence similarity between nucleic acids from said first organism and said second organism; providing a second substrate having a plurality of detection probes, wherein each detection probe is at a known location, and wherein at least one of said detection probes is complementary to a known nucleic acid sequence from said first organism and at least one of said detection probes is non-complementary to a known nucleic acid sequence from said first organism; contacting at least one sample nucleic acid from a third organism with said second substrate under conditions wherein when said at least one sample nucleic acid of said third organism is substantially complementary to a detection probe said at least one sample nucleic acid will preferentially hybidrize to a detection probe to which it is most complementary, resulting in at least one hybridized detection probe; determining a location of said at least one hybridized detection probe; identifying sequences of said at least one hybridized detection probe by referring to the location of said at least one hybridized detection probe; wherein when said sequence of said at least one hybridized detection probe is the same as a sequence complementary to said known nucleic acid sequence from said first organism, there is sequence similarity between nucleic acids from said first organism and said third organism; and identifying regions in said genome of said first organism where there is sequence similarity both with said nucleic acids from said second organism and said nucleic acids from said third organism, wherein said first organism and at least one of said second organism and said third organism diverged evolutionarily between about 60 million years ago and about 120 million years ago, and wherein regions in said nucleic acids from said first organism where there is sequence similarity with both said nucleic acids from said second organism and said third organism are candidate functional regions in said nucleic acid of said first organism.

In another specific embodiment of the invention there is a method for screening for genomic regions where polymorphisms have phenotypic effect in a first organism, comprising: providing a substrate having a plurality of detection probes, wherein each detection probe is at a known location, and wherein at least one of said detection probes is complementary to a known nucleic acid sequence from said first organism and at least one of said detection probes is non-complementary to a known nucleic acid sequence from said first organism; contacting at least one sample nucleic acid from a second organism with said substrate, where said second organism diverged evolutionarily from said first organism between about 60 million years ago and about 120 million years ago, and where said contacting is performed under conditions wherein when said at least one sample nucleic acid is substantially complementary to a detection probe said at least one sample nucleic acid will preferentially hybidrize to a detection probe to which it is most complementary, resulting in at least one hybridized detection probe; determining a location of said at least one hybridized detection probe; and identifying sequences of said at least one hybridized detection probe by referring to the location of said at least one hybridized detection probe; wherein when said sequence of said at least one hybridized detection probe is the same as a sequence complementary to said known nucleic acid sequence from said first organism, there is sequence similarity between nucleic acids from said first organism and said second organism, and regions in said nucleic acids of said first organism where there is sequence similarity with said nucleic acids from said second organism are regions where polymorphisms have phenotypic effect in a first organism.

In another aspect, methods are provided to screening for organism-differentiating regions of two organisms by comparing the genomic sequence from a first organism with the genomic sequence of a second organism without having to know the nucleic acid sequence from the second organism, where there is less than about 60 million years of evolutionary divergence between the first organism and the second organism. The method involves determining which bases from the nucleic acid from the second organism are identical to the bases from the nucleic acid of the first organism. The regions where the sequence diverges between the two organisms-i.e., the sequence similarity is below a pre-determined threshold value—are regions of putative organism-differentiating regions in both organisms. In the same way, the present invention allows for one to determine relative relatedness between organisms by using sequence comparison, where the sequence of only one organism needs to be known. The screening tests used herein will identify organism-differentiating regions and putative organism differentiating regions for further study.

A further specific embodiment of the invention is a method for screening for organism-differentiating sequences in nucleic acids of a first organism, comprising: providing a substrate having a plurality of detection probes, wherein each detection probe is at a known location, and wherein at least one of said detection probes is complementary to a known nucleic acid sequence from said first organism and at least one of said detection probes is non-complementary to a known nucleic acid sequence from said first organism; contacting at least one sample nucleic acid from a second organism with said substrate, where said second organism diverged evolutionarily from said first organism less than about 60 million years ago, and where said contacting is performed under conditions wherein when said at least one sample nucleic acid is substantially complementary to a detection probe said at least one sample nucleic acid will preferentially hybridize to a detection probe to which it is most complementary, resulting in at least one hybridized detection probe; determining a location of said at least one hybridized detection probe; and identifying sequences of said at least one hybridized detection probe by referring to the location of said at least one hybridized detection probe; wherein when said sequence of said at least one hybridized detection probe is the same as a sequence complementary to said known nucleic acid sequence from said first organism, there is sequence similarity between nucleic acids from said first organism and said second organism, and regions in said nucleic acids of said first organism where there is sequence divergence with said nucleic acids from said second organism are candidate organism-differentiating sequences in said nucleic acids of said first and second organisms.

Accordingly, the present invention also can be used to identify important polymorphisms and single nucleotide polymorphisms. The genomes of humans and other multicellular organisms contain a vast repository of intra-species polymorphic sites of which only a small proportion has functional significance. Some polymorphisms may lack functional significance because they occur within regions of the genome that themselves lack functional significance (e.g., certain intergenic regions). Other polymorphisms may occur in regions of the genome with functional significance; however, these polymorphisms do not affect a resulting amino acid sequence, change an amino acid sequence in a manner that has phenotypic effect, or are silent in non-coding regions with functional significance. The present invention provides methods for narrowing down the total repository of polymorphisms that need be analyzed for functionality, allowing one to focus on the smaller population of polymorphisms that are more likely to have phenotypic effects. The smaller population of polynucleotides are those occupying conserved regions between organisms. Accordingly, in another aspect of the invention, methods are provided to identify genomic rearrangements, including for example, deletions or insertions, by comparing the genomic sequence from a first organism with the genomic sequence of a second organism. The organisms may be from the same or from different species. The method comprises: providing a substrate having a plurality of detection probes, wherein each detection probe is at a known location, and wherein at least one of said detection probes is complementary to a known nucleic acid sequence from said first organism and at least one of said detection probes is non-complementary to a known nucleic acid sequence from said first organism; contacting at least one sample nucleic acid from a second organism with said substrate, where said contacting is performed under conditions wherein when said at least one sample nucleic acid is substantially complementary to a detection probe said at least one sample nucleic acid will preferentially hybridize to a detection probe to which it is most complementary, resulting in at least one hybridized detection probe; determining a location of said at least one hybridized detection probe; and identifying sequences of said at least one hybridized detection probe by referring to the location of said at least one hybridized detection probe; wherein when said sequence of said at least one hybridized detection probe is the same as a sequence complementary to said known nucleic acid sequence from said first organism, there is sequence similarity between nucleic acids from said first organism and said second organism, and regions in said nucleic acids of said first organism where there is sequence divergence with said nucleic acids from said second organism are rearrangement sequences in said nucleic acids of said first and second organisms. To confirm that these intervals of low conformance or sequence divergence correspond to sequence deletions, the method further comprises the step of preparing paired PCR primers to sequences bordering the intervals; using such primers, amplifying the nucleic acids of said first organism and said second organism; and comparing the length of the PCR products. If the PCR product resulting from the first organism is longer than that from the second organism, the interval corresponds to a deletion in the second organism.

### EXAMPLES

1. Evolutionarily Conserved Sequences on Human Chromosome 21 by Comparing Human and Dog and Human and Mouse Sequences

Human chromosome 21 was examined for evolutionarily conserved elements by hybridization of mouse and dog bacteria artificial chromosome (BAC) sequences to human oligonucleotide arrays. For cross-species comparisons, the sequences should be orthologous (derived from the same piece of DNA) and not paralogous (similar due to a duplication of DNA). If paralogous sequences between two species are compared, the number of conserved elements can be underestimated. In this study, mouse and dog BACs were considered orthologous if they contained two or more markers present on human chromosome 21 (comparative anchor tag sequences (CATS)) and formed part of a contig. In addition, BACs identified by a single marker, such as those at the edge of a contig or in a region not spanned by a contig, were considered orthologous if extended regions of conservation outside of known coding sequences were observed when they were hybridized to the oligonucleotide arrays.

Orthologous chromosome 21 sequences were isolated using CATS to coding and non-coding conserved elements. 106 human chromosome 21 segments were obtained through (http://www.ncbi.nlm.nih.gov/genome/seq/chr.cgi? CHR=21&SRT=size&MIN=0&ORG=Hs), masked for repeats using RepeatMasker2 (A. F. A. Smit & P. Green, supra) and queried against the Mouse BAC End (at ftp.tigr.org/pub/data/m_musculus/bac_end_sequences/), GenBank nt and dbEST (restricted to the mouse) databases using BLAST (S. F Altschul, supra). Matches between chromosome 21 DNA and sequences in the Mouse BAC End (with an E value≦10⁻¹⁰) and GenBank (to known or suspected mouse orthologs) databases were used to design CATSs (with ˜50% GC content and a predicted product of 100-200 basepairs). Each primer pair was individually tested against human and mouse genomic DNA to determine if the pair produced a single clear fragment visible by agarose gel electrophoresis and ethidium-bromide staining. All mouse-specific primers used in the study were obtained from either the Mouse Genome Database (http://www.informatics.jax.org/) or the WICGR Mouse RH Map (http://www.genome. wi.mit.edu/mouse_rh/index.html). A total of 123 CATS were developed. These markers along with mouse-specific syntenic markers were used to screen the RPCI-23 mouse BAC library by the polymerase chain reaction (PCR).

PCR reactions were performed in a 25-μl volume containing 10 ng of genomic DNA or 1 ng of purified BAC DNA, 1 mM of each primer, 2.5 units of AmpliTaq Gold (Perkin-Elmer), 0.25 mM deoxynucleotide triphosphates (dNTPs), 10 mM tris-HCl (pH 8.3), 50 mM KCl, and 1.25 mM MgCl₂. Thermocycling was performed on a 9600 or 9700 (Perkin-Elmer), with initial denaturation at 95° C. for 10 min, followed by one of two cycling conditions based on the melting temperature of the primers: either 10 cycles of [94° C. 30 sec, 58° C. 30 sec, 72° C. 30 sec] followed by 30 cycles of [94° C. 30 sec, 55° C. 30 sec, 72° C. 30 sec] or 10 cycles of [94° C. 30 sec, 55° C. 30 sec, 72° C. 30 sec] followed by 30 cycles of [94° C. 30 sec, 52° C. 30 sec, 72° C. 30 sec]. A final extension reaction was carried out at 72° C. for 5 min. To score BACs for the presence or absence of markers, 10-μl of the PCR amplification product was assayed by 2% agarose gel electrophoresis and ethidium-bromide staining.

These efforts combined with existing mouse maps (see T. Wiltshire, et al., Genome Res. 9:1214 (1999) and M. Pletcher, et al., Genomics, submitted) resulted in the assembly of >360 mouse BACs and plasmid artificial chromosomes (PACs) into 35 contigs which span ˜74% of the syntenic human chromosome 21 sequences.

A 6-Mb 21q22 region, known as the Down Syndrome Critical Region, was targeted for human-dog analysis because of the intense biological interest in this interval. Twenty-one CATS spanning the 6-Mb 21q22 interval were amplified from dog genomic DNA by PCR and used to screen the RPCI-65 dog BAC library by hybridization. Sixty-one dog BACs were isolated, characterized by PCR content mapping, and assembled into 9 contigs covering 4 Mb (67%) of the targeted syntenic chromosome 21 region.

Human chromosome 21 sequence was used to design high-density arrays consisting of 25-mer oligonucleotides (probes) (see, for example, FIGS. 1-3) (for methods, see, for example, M. Chee, et al., Science 274: 610 (1996); S. P. Fodor, et al., Science 767 (1991); A. C. Pease, et al., Proc. Natl. Acad. Sci. USA 91:5022 (1994)) and WO 95/11995, WO 92/10092, or U.S. Pat. Nos. 5,143,854; 5,384,261; 5,405,783; 5,412,087; 5,424,186; 5,445,934; 5,744,305; 5,800,992; 6,040,138; 6,040,193, all of which are incorporated herein by reference for all purposes).

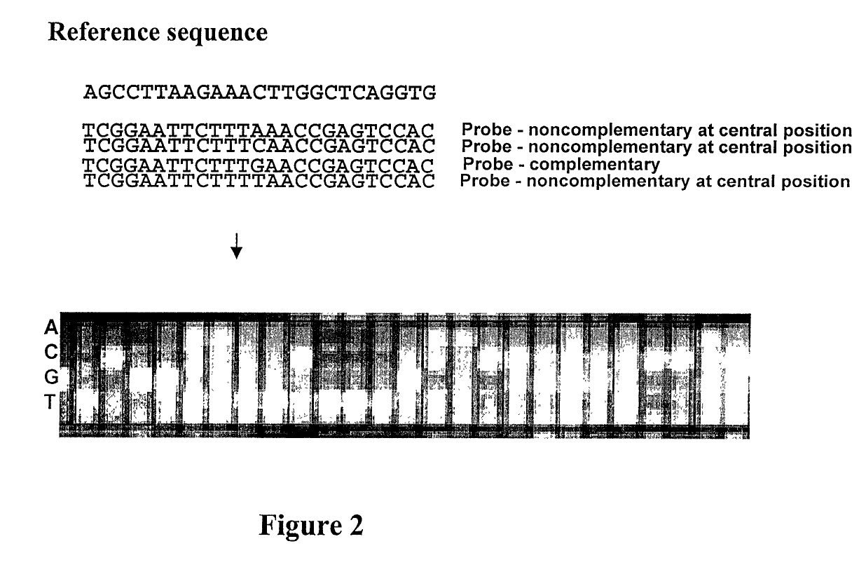

Four probes were designed to interrogate each nucleotide present on each strand of chromosome 21 sequence, one probe complementary to the sequence and three mismatch probes identical to the complementary probe except for the nucleotide at the central position (the 13^{th} position) under interrogation. At this central position, each mismatch probe contains one of the bases not identical to the perfect match probe. When the fluorescence intensities (white squares) of the complementary probes are greater than that of the non-complementary probes, similarities between the tiled human sequences and the hybridized animal DNA exist. See, FIG. 2. In this study ˜276 arrays containing greater than 130 million oligonucleotides were used to interrogate ˜33-Mb of non-repetitive chromosome 21 sequence (˜16.5-Mb of each Watson and Crick complementary strands).

DNA labeling and hybridization to arrays was performed as described in D. G. Wang et al., Science 280: 1077 (1998) with minor modifications. 30 μg of purified BAC DNA was fragmented with deoxyribonuclease (DNase) 1 (Boehringer Manneheim) and labeled with biotin with terminal deoxynucleotidyl transferase (TdT, GibcoBRL Life Technology). Fragmentation was performed in a 74-μl volume with 0.2 unit of DNase 1, 10 mM tris-acetate (pH 7.5), 10 mM magnesium acetate, and 50 mM potassium acetate at 37° C. for 10 min, after which the reaction was stopped by heat inactivation at 99° C. for 10 min. The terminal transferase reaction was performed by adding 50 units of TdT and 12.5 μM biotin-N6-ddATP (Dupont NEN) to the preceding reaction mix, incubating at 37° C. for 90 min, and then heat-inactivating at 99° C. for 10 min. Next, labeled DNA sample was denatured in hybridization buffer [3M tetramethylammonium chloride, 10 mM tris-HCl (pH 7.8), 0.01% Triton X-100, herring sperm DNA (100 μg/ml), and 50 pM control oliogomer] at 99° C. for 5 min and hybridized to an oligonucleotide array overnight at 40° C. on a rotisserie at 40 rpm. All washes and staining were performed at room temperature. Oligonucleotide arrays were washed twice with 1×MES buffer [0.1 M 2-[N-Morpoline]ethanesulfonic acid (pH 6.7), 1 M NaCl, and 0.01% Triton X-100], and stained with staining solution [streptavidin R-phycoerythrin (20 μg/ml) (Molecular Probes) and acetylated bovine serum albumin (BSA) (1 mg/ml) in 2×MES] for 20 min on a rotisserie at 40 rpm. Following two washes with 1×MES, chips were incubated with antibody solution [biotinylated anti-streptavidin antibody (10 μg/ml) and BSA (1 mg/ml) in 2×MES] for 20 min on a rotisserie at 40 rpm. After two washes with 1×MES, chips were stained again with staining solution for 20 min. Oligonucleotide arrays were washed 6 times with 6×SSPET [0.9 M NaCl, 60 mM NaH₂PO₄, 6 mM EDTA (pH 7.4), 0.01% Triton X-100] at 35° C. on a fluidics workstation (Affymetrix). Hybridization was detected by using a custom confocal scanner with a resolution of 110 pixels per feature (pixel size of 2.27 μM) and 560-nm filter.

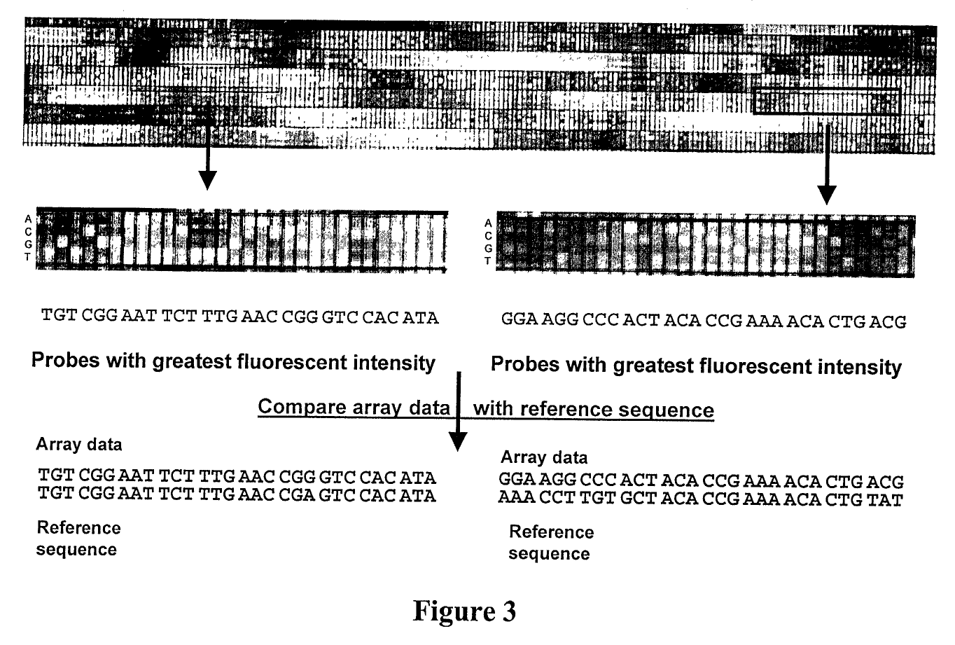

The chromosome 21 arrays were designed using non-repetitive sequences and hybridized with syntenic mouse and dog BACs that are represented as horizontal lines. A low magnification view of fluorescence hybridization image of an array is shown in FIG. 1. Two 30 nucleotide intervals, one with high conformance between the human and dog sequences (left rectangle in array display) and one with low conformance between human and dog sequences (right rectangle in array display), are shown in FIG. 3. The conserved sequence with high conformance (97%) shows the 29 conforming nucleotides. The conserved sequence with low conformance (60%) of 18 conforming nucleotides is also shown.

Data from the probe arrays were used to identify regions as potentially conserved between species. The identification procedure was developed by inspecting CONSEQ graphs of mouse DNA. This program allowed conformance to be computed over varying window sizes, with different amounts of overlap between the windows. CONSEQ also allowed the scientists to examine the reference sequence, the called cross-species sequence, and the location of repeats that were tiled on the probe arrays. Based on this evidence, it was determined that windows of 30 base pairs with an overlap of 10 base pairs that had conformance of 60% or higher showed strong similarity between human and mouse, except in cases where the window was close to or within a repeat or a region of low complexity.

By inspection, it was determined that windows that were within 20 base pairs of a repeat sometimes showed spuriously high conformance. Likewise, inspection of windows with high conformance led to the decision that windows in which the reference sequence had either (a) 50% or more of a single base (either A, G, T, or C) or (b) 67% or more of a single base within any sub-window of 15 base pairs within the 30-base pair window (i.e., 10 or more out of 15), would sometimes show high conformance. Such sequences of low local complexity were considered not of interest, and were therefore not classified as potentially conserved. Finally, inspection of CONSEQ graphs led to the conclusion that nearby windows with high conformance were likely to be parts of the same potentially-conserved element. For example, there were clear cases where an exon was conserved and most, but not all, windows covering that exon showed high conformance. Thus, it was determined that regions of 120 base pairs or less between potentially conserved-windows would also be declared as potentially conserved.

The procedure for determining potentially conserved-regions was a multi-step process. The first step computed conformance for all 30-base pair windows (with 10-base pair overlap). In other words, the conformance was computed for base pairs 1-30, 21-50, 41-70, and so on, as the percent of probes matching the reference sequence (of the 60 probes—30 for the Watson strand, 30 for the Crick strand). Next, the distance of each window from the nearest known repeat was computed, using the output from RepeatMasker run on the reference sequence. Then the maximum frequency of any base in the reference sequence corresponding to each window was computed. For example, if in the first 30 base pairs of the reference sequence there were 10 A's, 8 C's, 7 G's, and 5 T's, then the maximum frequency would be 10. Finally, the maximum frequency of any base within a sub-window of 15 base pairs within the reference sequence was computed for each window. For the first window (base pairs 1-30 of the reference sequence), the 16 sub-windows would be base pairs 1-15, 2-16, . . . , 16-30; within each of the 16 sub-windows, the maximum frequency of any single base was computed, then the final result was the maximum of those 16 values. After these statistics were computed, windows were classified as potentially conserved for which (a) conformance was at least 60%, (b) nearest repeat was more than 20 base pairs away, (c) maximum single-base frequency was less than 50%, and (d) maximum single-base frequency for any 15-base pair sub-window was less than 67%. Then, for all potentially-conserved windows within 120 base pairs of another potentially-conserved window, the windows between them were also classified as potentially conserved. So, for example, if the window from base pairs 41-70 was potentially conserved, and the next potentially-conserved window was from base pairs 161-190, the windows at base pairs 61-90, 81-110, . . ., and 141-170 were also classified as potentially conserved. Finally, from the collection of potentially-conserved windows, the potentially-conserved contiguous regions were computed. Thus, if windows from base pairs 201-230, 221-250, and 241-270 were potentially conserved (but windows before and after were not), the region from base pairs 201-270 was classified as potentially conserved.

Once the identity parameters were determined, labeled mouse and dog sequences were incubated with the arrays. If the perfect match probe had greater fluorescent intensity than the corresponding mismatch probes, the nucleotide under interrogation was referred to as “conforming” to the human reference sequence. To identify conserved regions, 30-nucleotide (nt) windows (with 10 nt overlap with neighboring windows) were examined and the conformance of the Crick and Watson strands were averaged. For example, if in a 30-nt window 75% of the Crick strand nucleotides and 85% of the Watson strand nucleotides conformed to the reference sequence, the window would have a reported conformance of 80%.

Empirically-derived criteria were used to define a conserved element as a sequence with >60% conformance and >30 bp in length. The goal was to develop stringent criteria so that the resulting set of conserved elements would have high specificity (low false positive rate) with correspondingly lower sensitivity (higher false negative rate).

To estimate the false positive rate, 10 chromosome 21 arrays (600 kb) were hybridized with non-orthologous mouse DNA. Only 7 of the 30-nt windows had a conformance of ≧60%, of which 3 were low complexity sequences (a high percentage of a single base). Based on these results low complexity sequences were excluded as conserved elements. The same 600 kb segment of chromosome 21 sequence was hybridized with orthologous mouse DNA and by comparing the number of base pairs called conserved with non-orthologous versus orthologous mouse DNA, the false positive rate was estimated to be ˜1%. When these rules were used to analyze 4 arrays containing ˜240 kb of chromosome 21 sequence hybridized with non-orthologous dog DNA, not a single 30-nt window was identified as conserved.

The false negative rate was estimated by determining the percentage of exons the arrays failed to detect for twenty-two chromosome 21 genes with known mouse orthologs that have previously been sequenced. Human chromosome 21 sequence was searched against the GenBank database (Nov 2000) restricted to mouse using BLAST (default parameters). The matches of the following genes were inspected to ensure that only those corresponding to human-mouse orthologs were used: SAMSN-1, CXADR, BTG3, PRSS7, NCAM2, GABPA, APP, CCT8, BACH1, CLDN8, IFNAR2, IL10RB, GART, CBR1, CLDN14, SIM2, DSCAM, BACE2, PKNOX1, PFKL, SMT3H1, COL6A2. Exonic sequences in regions not analyzed by the oligonucleotide arrays were not used to calculate the false negative rate. The twenty-two genes were chosen to represent coding elements along the entire length of chromosome 21 with varying degrees of similarity between the human and mouse orthologs. One hundred and ninety exons had electronic matches using the BLAST algorithm and a cutoff of E≦10⁻⁵ (where E is the expected value). After hybridizing the mouse BACs with the arrays and analyzing the data, 74% of the 190 electronic matches were identified as conserved elements in the analysis (see Table 1).

TABLE 1

| | # of | % | | Total bp | | Array |
|---|---|---|---|---|---|---|
| | BLAST | identified | BLAST | (%) | BLAST | % |
| Expect score | matches | by array | length (bp) | overlap | % ID | CON |
| 10⁻¹⁰ to 10⁻⁰⁵ | 20 | 50 | 73 | 658 (42) | 88 | 71 |
| 10⁻²⁰ to 10⁻¹⁰ | 47 | 55 | 90 | 2359 (41) | 89 | 72 |
| 10⁻³⁰ to 10⁻²⁰ | 40 | 72 | 126 | 3472 (45) | 89 | 72 |
| 10⁻⁴⁰ to 10⁻³⁰ | 24 | 79 | 151 | 2799 (51) | 89 | 68 |
| 10⁻⁶⁰ to 10⁻⁴⁰ | 29 | 90 | 169 | 4390 (54) | 90 | 69 |
| less than 10⁻⁶⁰ | 30 | 100 | 322 | 9652 (49) | 90 | 65 |
| Total | 190 | 74 | 152 | 23330 (49) | 89 | 69 |

Table 1 provides an estimation of the false negative rate. The electronic matches of 190 exons were divided into 6 classes based on their Expect scores. # of BLAST matches=the number of electronic matches in the class, % identified by array=the percent of electronic matches in the class that were identified as conserved elements by the array analysis, BLAST length (bp)=the mean length in base pairs of the electronic matches in the class, Total bp (%) overlap=for the conserved elements identified by both BLAST and the array−the total number of base pairs in the electronic matches and the percent of those base pairs identified by the array, BLAST % ID=the mean percent identity of the electronic matches in class, Array % CON=the mean percent conformance of the base pairs identified by both BLAST and the array.

The majority of the electronic matches missed were short (mean BLAST length≦90 bp); only 54% of the matches with E≧10⁻²⁰ were identified versus 85% of the matches with E≦10⁻²⁰. These data were also used to gauge how percent conformances and lengths of conserved elements identified by arrays compare with percent identities and lengths of conserved elements identified by sequence alignments. For the 140 conserved elements found by both BLAST and array analyses, the mean percent identities and percent conformances were 89% and 69%, respectively. Forty-nine percent of the base pairs present in the 140 electronic matches were represented in the conserved elements identified by the arrays. Thus the stringent criteria used in this analysis to minimize the number of false positives results in an underestimation of the number of conserved human-mouse elements and the elements that are found are shorter in length than those identified by sequence alignments. Chromosome 21 sequence and biological annotations were retrieved from GenBank in 106 segments, most of which are 340 kb size and have 1-kb overlap with neighboring segments (M. Hattori et al., Nature 405:311 (2000)).

All of chromosome 21-sequence, except for interspersed repeats identified by RepeatMasker was tiled on the arrays. The percentage of human chromosome 21 analyzed was defined as the number of tiled base pairs hybridized to orthologous mouse DNA (16,580,114), divided by the total number of non-repetitive base pairs tiled on the arrays (22,490,347)=˜74%. In the ˜74% of chromosome 21 analyzed by hybridization with orthologous mouse DNA, the arrays identified 3,398 conserved elements, of which 895 overlapped exons of known 21 q genes (as annotated in GenBank files). The identified elements hybridized with mouse DNA, are noncontiguous and span ˜30 Mb. The unidentified, conserved remaining 2,503 elements were examined to determine if they had similarities to known exonic sequences: 135 were exons of chromosome 21 genes (missing GenBank annotations), 34 matched genes not previously assigned to chromosome 21, and 77 matched ESTs (many are likely alternatively spliced exons). The remaining 2,257 were not in identified exons (NIEs). In the segment of chromosome 21 analyzed, ˜1.6% of the base pairs outside of repetitive elements are conserved (260,226 bp) of which 56% corresponds to the 2,257 NIEs and 44% corresponds to the 1,141 identified exons (IEs).

TABLE 2

| # of | # of | % of | Length (bp) | | | |
|---|---|---|---|---|---|---|
| elements | bps | hyb'd bps | Mean | S.D. | Min. | Max. |

≧30 bp

| | | | | | | | |
|---|---|---|---|---|---|---|---|
| Total | 3398 | 260226 | 1.6 | 76 | 109 | 30 | 2690 |
| NIE | 2257 | 145010 | 0.9 | 64 | 78 | 30 | 950 |
| IE | 1141 | 115216 | 0.6 | 101 | 150 | 30 | 2690 |
| ≧50 bp | | | | | | | |
| Total | 1478 | 202623 | 1.2 | 137 | 144 | 50 | 2690 |
| NIE | 762 | 100160 | 0.7 | 131 | 105 | 50 | 950 |
| IE | 716 | 102463 | 0.6 | 143 | 176 | 50 | 2690 |

Table 2 shows the number and sizes of human-mouse conserved elements. ≧30 bp=analysis of all elements fitting criteria of conservation; ≧50 bp=analysis of the subset of conserved elements that are greater than or equal to 50 base pairs in length; Total=both NIE and IE classes; # of elements=number of conserved elements identified; # of bps=the number of base pairs covered by all the conserved elements; % of hyb'd bps=the percent of the hybridized tiled base pairs which are conserved. For the length data: Mean=mean length in base pairs of conserved elements, S.D.=standard deviation, Min.=length of the shortest element, Max.=length of the longest element. For detailed analysis, see Table 3.

Since long human-mouse elements are more likely to be actively conserved than shorter ones, the set of elements analyzed were those≧50 nucleotides in length. Although this represents only 43% of all human-mouse elements because those eliminated were short, the amount of chromosome 21 sequence considered conserved is only reduced by 22%. In this set of longer elements, the numbers and lengths of the NIEs and IEs is similar (Table 3). These data suggest that known genes compose only half of the sequences on chromosome 21 conserved between humans and mice.

Chromosome 21 contains 225 genes, of which 127 correspond to known genes and 98 represent genes predicted in silico. These predictions were compared to the human-mouse conservation results obtained by the methods of the present invention. Sixty-nine predictions were examined; 14 of the 15 class 1 (those with similarity to a previously identified gene or ORF) and 13 of the 54 class 2 (those based solely on spliced EST matches and/or consistent exon predictions) predictions had at least one exon conserved. These results indicate that class 1 predictions are supported by human-mouse conservation whereas the majority of class 2 predictions are not.

The distribution of conserved human-mouse sequences on chromosome 21 was examined by calculating the percent of base pairs conserved in consecutive 300-kb intervals with 1-kb overlaps. The number of base pairs conserved in the intervals ranged from 0.1-4.16%. For the two intervals with the highest levels of conservation, one was dominated by IE elements and the other by NIE elements. These data suggests that the percentage of base pairs conserved in the 300-kb intervals is not directly correlated with known coding potential.

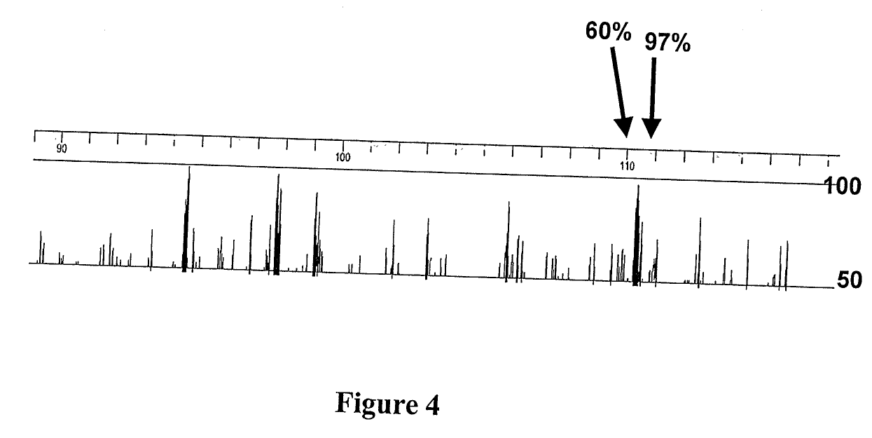

In the ˜12% of 21q sequence hybridized with orthologous dog DNA (the number of tiled base pairs hybridized to orthologous dog DNA (2,597,732) divided by the total number of non-repetitive chromosome 21 base pairs tiled on the arrays (22,490,347)=12%), 1,292 conserved elements were identified. Of these, 240 are IE and 1,052 are NIE elements. The arrays identified 1,292 conserved human-dog elements of which 197 overlapped exons of known chromosome 21 genes (as annotated in GenBank files). The remaining 1,095 conserved elements were compared against the GenBank nt (Nov 2000) and dbEST (Jan 2001) databases using BLAST (default parameters). Matches with expect values ≦10⁻⁵ and the words “genomic DNA” or “Chromosome 21” in the FASTA description line were excluded. Of the 1,095 elements, 10 were exons of known chromosome 21 genes (missing GenBank annotations), and 14 matched cDNAs not assigned to chromosome 21 at the time the sequence was released. FIGS. 3 and 4 show data obtained from human chromosome 21 sequence hybridized with syntenic dog sequence. FIG. 3 shows an enlarged view of a human 21q array hybridized with syntenic dog BAC DNA (top). Two 30 nucleotide intervals, one with high conformance between the human and dog sequences (left rectangle) and one with low conformance between human and dog sequences (right rectangle), are shown. For the conserved sequence with high conformance (97%), the 29 conforming nucleotides are shown. For the conserved sequence with low conformance (60%), the 18 conforming nucleotides are shown. FIG. 4 shows a CONSEQ plot of conserved regions identified by hybridization with syntenic dog sequences for a 26-kb interval on chromosome 21. Conserved elements (highlighted peaks) detected are shown relative to their position in the human reference sequence (horizontal axis), and their percent conformance (50-100%) is indicated on the vertical axis. The high conformance (97%) conserved sequence has been merged with neighboring conserved sequences to form a 200-nt conserved element. The low conformance (60%) conserved sequence is a 30-nt element. Small rectangles on the top line indicate the positions of interspersed repeats, which were not tiled on the arrays, therefore conformance information is absent.

The 21q.22 region hybridized with both mouse and dog DNA (˜10% of 21 q) was used to compare the human-mouse and human-dog conserved elements. (The number of tiled base pairs hybridized to both orthologous mouse and dog DNA (2,232,610), divided by the total number of non-repetitive chromosome 21 base pairs tiled on the arrays (22,490,347)=˜10%. These base pairs are noncontiguous and span ˜6 Mb in the 22q.22 region.) In this region, ˜4.3% and ˜1.3% of the base pairs outside of repetitive elements were conserved in the dog and mouse analyses, respectively (Table 3).

TABLE 3

| | IE | | | | | | NIE | | | | | |
|---|---|---|---|---|---|---|---|---|---|---|---|---|
| | | % of | Length | | | | | % of | Length | | | |
| | (n) | hyb'd bps | Mean | S.D. | Min. | Max. | (n) | hyb'd bps | Mean | S.D. | Min. | Max. |
| Total Dog | 219 | 1.1 | 112.3 | 108.9 | 30 | 710 | 956 | 3.2 | 74.6 | 94.1 | 30 | 1,250 |
| Dog/Mouse | 132 | 0.8 | 137.6 | 125.4 | 30 | 710 | 114 | 1.0 | 196.2 | 186.0 | 30 | 1,250 |
| Dog only | 87 | 0.3 | 73.9 | 60.5 | 30 | 370 | 842 | 2.2 | 58.2 | 56.0 | 30 | 410 |
| Total Mouse | 140 | 0.5 | 79.1 | 81.9 | 30 | 670 | 240 | 0.7 | 63.0 | 85.4 | 30 | 950 |
| Mouse/Dog | 129 | 0.5 | 81.0 | 84.0 | 30 | 670 | 120 | 0.5 | 90.7 | 113.2 | 30 | 950 |
| Mouse only | 11 | 0.0 | 57.3 | 47.6 | 30 | 190 | 120 | 0.2 | 35.3 | 16.9 | 30 | 130 |

Table 3 shows a comparison of the number and lengths of human/dog and human/mouse conserved elements identified in ˜10% of chromosome 21. Total Dog=all the human/dog elements; Dog/Mouse=the human/dog elements that overlap human/mouse elements; Dog only=the human/dog elements that do not overlap human/mouse elements; Total Mouse=all the human/mouse elements; Mouse/Dog=the human/mouse elements that overlap human/dog elements; Mouse only=the human/mouse elements that do not overlap human/dog elements. The number of conserved elements identified (n) and the percent of the hybridized non-repetitive base pairs (% of hyb'd bps) covered by all the conserved elements, is given. The number of elements in the Dog/Mouse and the Mouse/Dog groups are different because multiple elements in one analysis are equal to one element in the other. For the length data: Mean=the mean length in base pairs of all conserved elements, S.D.=standard deviation of length, Min.=length of the shortest element, Max.=length of the longest element.

The dog analysis identified considerably more IEs and NIEs than the mouse analysis. The conserved elements (IEs and NIEs) identified in both analyses are usually longer suggesting a higher level of conservation than those identified in a single species. Unlike IEs that have clear function, the function of NIEs is unclear. NIEs present in all three species (human/dog/mouse), however are more likely to be conserved due to functional constraints than NIEs observed in only two species.

2. Evolutionarily Conserved Sequences on Human Chromosome 21 by Comparing Human Sequence to Primate Sequence

Human chromosome 21 was examined for evolutionarily conserved elements by hybridization of gorilla, chimpanzee and macaque sequences to human oligonucleotide arrays. Unlike the dog and mouse nucleic acid samples, the primate nucleic acid samples were prepared by long range PCR amplification of genomic DNA. Protocols much like the following were employed. Primers used for the amplification reaction were designed in the following way: a human chromosome 21 sequence was fed into the software program Repeat Masker which recognizes sequences that are repeated in the genome (i.e., Alu and Line elements). The repeated sequences are “masked” by the program by substituting the specific nucleotides of the sequence (A, T, G or C) with “Ns”. The sequence output after this repeat mask substitution was then fed into a commercially available primer design program (Oligo 6.23) to select primers that were greater than 30 nucleotides in length, had melting temperatures of over 65° C. and had sequences chosen only from the non-repetitive regions. The designed primer output from Oligo 6.23 was then fed into a program which then “chose” primer pairs which would PCR amplify a given region of the genome but have minimal overlap. An illustrative protocol for long range PCR is as follows:

Reagents Used:

1. Expand™ Long Template PCR System from Boehringer Mannheim Cat.# 1681 834, 1681 842, or 1759 060.

2. 100 mM dNTP set from Life Technologies, Cat.# 10297-018.

3. Molecular Biology Grade Water from Bio Whittaker, Cat.# 16-001Y.

4. 1 M MgCl₂ from Sigma, Cat.# M 1028.

2 master mixes are required for each 50 μL PCR reaction:

Separate Master Mix 1 was prepared for each template in 1.5 ml microfuge tubes on ice:

1. Master mix 1 (for 1 PCR reaction)

Add Bio Whittaker water to a final volume of 19 μL

2.5 μL 10 mM dNTP mix (containing dATP, dCTP, dGTP, and dTTP at 10 mM each) for a final concentration of 500 μM each dNTP

50 ng DNA template

2. Master Mix 2 for all reactions (+1 extra) was then prepared and kept on ice:

Master mix 2 (for 1 PCR reaction)

Add Bio Whittaker water to a final volume of 25 μL

5 μL 10×PCR buffer 3 (which contains 22.50 mM MgCl₂)

2.5 μL 10 mM MgCl₂ (for a final MgCl₂ concentration of 2.75 mM)

0.75 μL enzyme mix (add last)

Six microliters of premixed primers (containing 2.5 μM of each primer) were added to 8 strip PCR tubes on ice. Next, 19 μL of Master Mix 1 was added to appropriate tubes, then 25 μL of Master Mix 2 was added to each tube. The tubes were capped, mixed, centrifuged briefly and returned to ice. At this point, the PCR cycling was begun according to the following program: step 1: 94° C. for 3 min to denature template; step 2: 94° C. for 30 sec; step 3: annealing for 30 sec at a temperature appropriate for the primers used; step 4: elongation at 68° C. for 1 min/kb of product; step 5: repetition of steps 2-4 38 times for a total of 39 cycles; step 6: 94° C. for 30 sec; step 7: annealing for 30 sec; step 8: elongation at 68° C. for 1 min/kb of product plus 5 additional minutes; and step 9: hold at 4° C. Alternatively, a two-step PCR would be performed: step 1: 94° C. for 3 min to denature template; step 2: 94° C. for 30 sec; step 3: annealing and elongation at 68° C. for 1 min/kb of product; step 4: repetition of steps 2-3 38 times for a total of 39 cycles; step 5: 94° C. for 30 sec; step 6: annealing and elongation at 68° C. for 1 min/kb of product plus 5 additional minutes; and step 7: hold at 4° C.

Human chromosome 21 sequence was used to design high-density arrays consisting of 25-mer oligonucleotides (probes) (see, for example, M. Chee, et al., Science 274: 610 (1996); S. P. Fodor, et al., 767 (1991); A. C. Pease, et al., Proc. Natl. Acad. Sci. USA 91:5022 (1994)) and WO 95/11995, WO 92/10092, or U.S. Pat. Nos. 5,143,854; 5,384,261; 5,405,783; 5,412,087; 5,424,186; 5,445,934; 5,744,305; 5,800,992; 6,040,138; 6,040,193, all of which are incorporated herein by reference for all purposes). Four probes were designed to interrogate each nucleotide present in chromosome 21 sequence, one probe complementary to the sequence and three mismatch probes identical to the complementary probe except for the nucleotide at the central position (the 13^{th} position) under interrogation. At this central position, each mismatch probe contains one of the bases not identical to the perfect match probe.

DNA labeling and hybridization to arrays was performed as described in D. G. Wang et al., Science 280: 1077 (1998) with minor modifications. The amplified genomic DNA was fragmented with deoxyribonuclease (Dnase) 1 and labeled with biotin with terminal deoxynucleotidyl transferase as described in the first Example. Next, labeled DNA samples were denatured in hybridization buffer and hybridized to an oligonucleotide array overnight at 40° C. on a rotisserie at 40 rpm. Hybridization was detected by using a custom confocal scanner with a resolution of 110 pixels per feature (pixel size of 2.27 μM) and 560-nm filter.

If, upon incubation of the labeled gorilla, chimp or macaque samples with the arrays the perfect match probe had greater fluorescent intensity than the corresponding mismatch probes, the nucleotide under interrogation was referred to as “conforming” to the human reference sequence. To identify conserved regions, 30-nucleotide (nt) windows (with 10 nt overlap with neighboring windows) were examined and the conformance of the Crick and Watson strands were averaged. For example, if in a 30-nt window 75% of the Crick strand nucleotides and 85% of the Watson strand nucleotides conformed to the reference sequence, the window would have a reported conformance of 80%.

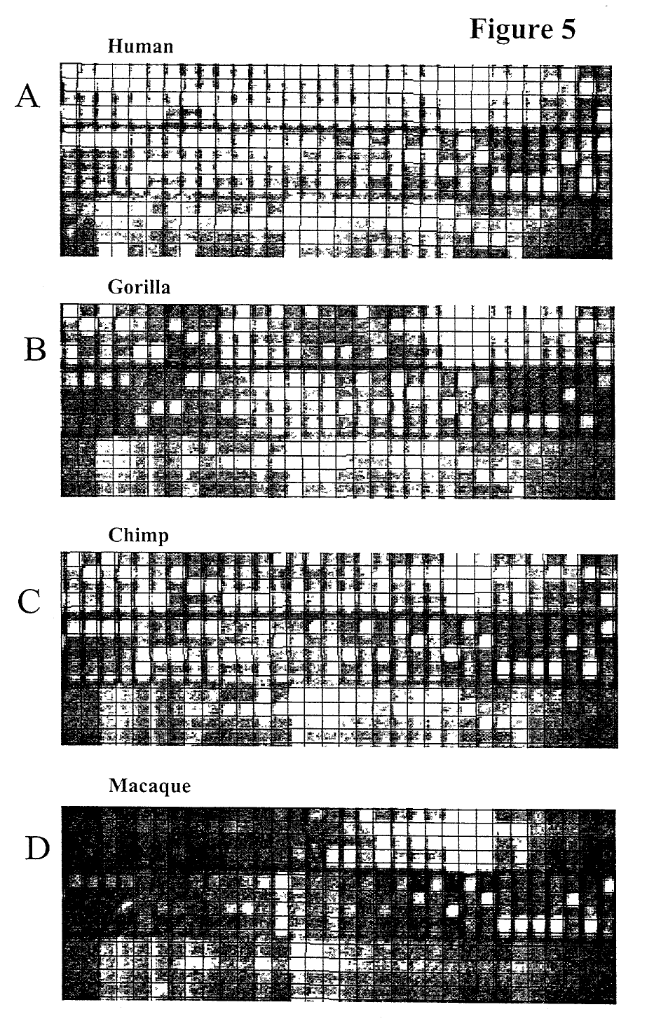

The results of scans performed on four substrate-bound oligonucleotide arrays are shown in FIG. 5. The sequence of the probes on these arrays is based on human genomic sequence from chromosome 21. Four identical arrays were hybridized with human, gorilla, chimpanzee or macaque amplified genomic DNA samples. Each column of the array has a group or set of four probes, each probe having a different base in the interrogation position. The sequence of the base in the interrogation position is, from top to bottom, A-C-G-T. A “street” or unoccupied position is inserted in the column in the fifth position, then another set of four probes occurs. In this set of four probes, the same scheme is used; each probe has a different base in the interrogation position and the sequence of the base in the interrogation position is, from top to bottom, A-C-G-T, a street position is inserted and so on. The horizontal rows correspond to the reference sequence as described above. In looking at the scans, one can see that the pattern of hybridization is very similar between the human, gorilla and chimp sequences. The patterns of hybridization of the human and macaque samples have enough similarity to detect conserved bases, but the sequence divergence is becoming more pronounced. Also this data shows that sequence can be determined quickly in regions of both the gorilla and the chimp genomes. Thus, the present invention is useful for rapid sequencing of regions of high conformance between sequences when one of the sequences is known.

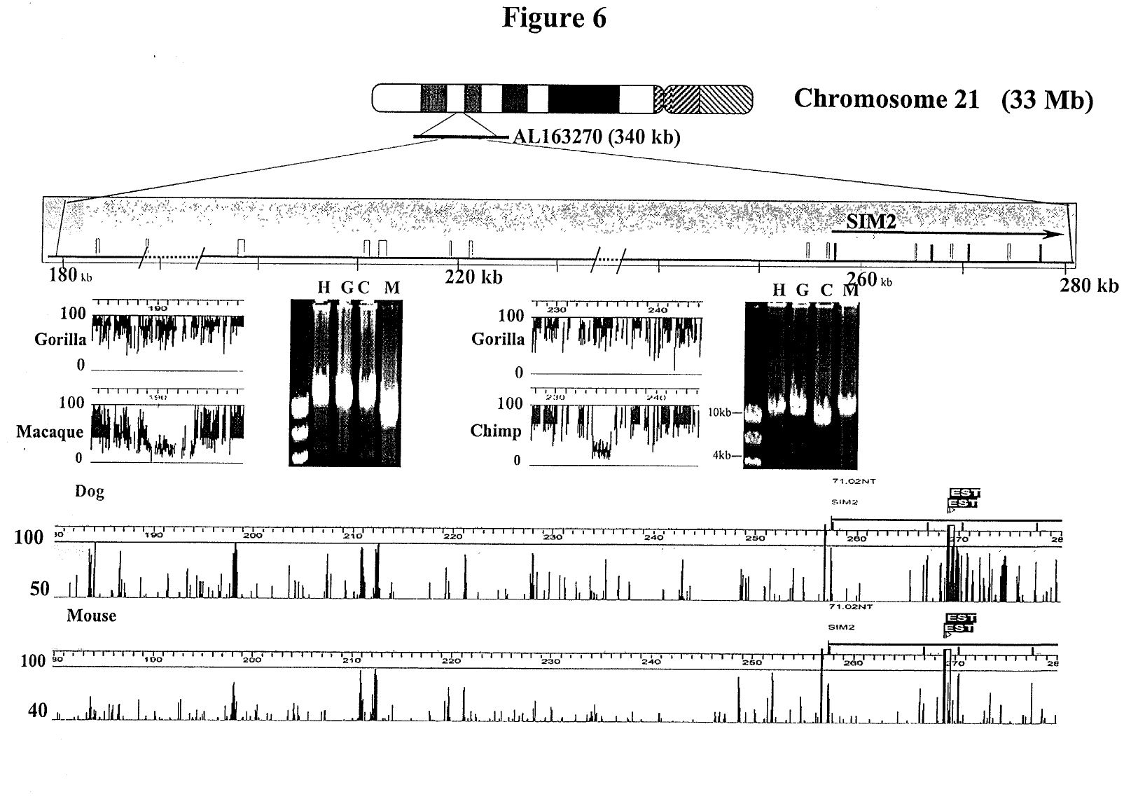

Detailed results of a 100 kb interval of the SIM2 region of human chromosome 21 is seen in FIG. 6. Mouse and dog CONSEQ plots are shown at the bottom of the figure. Conserved elements are highlighted relative to their position in the human reference sequence horizontal axes, and their percent conformance (0-100%) are indicated on the vertical axes. Peaks with≧60% conformance are shown. Shaded peaks not highlighted have≧60% conformance but are low complexity or are close to a repeat. The locations of GenBank annotated single-minded 2 (SIM2) exons (rectangles), elements identified as coding sequences by database searches (white rectangles with black outline), and chromosome 21 cross-species markers (black with highlighted background) are shown. Small rectangles at the top line of the plots indicate the positions of interspersed repeats, which were not tiled on the arrays, and therefore conformance information is absent. Note that the baseline for sequence similarity in the plot for dog is set at 50% and for mouse is set at 40%.

Note that the baseline for sequence similarity in the plot for dog is set at 50% and for mouse is set at 40%. In addition to the dog and mouse plots, CONSEQ plots of conserved regions identified by hybridization with amplified gorilla, macaque and chimp sequences for the >10 kb intervals indicated are shown. In these primate plots, the baseline for sequence similarity is set at 0%. Photographs of the agarose gels of the genomic DNA amplified each primate from the region indicated are also shown.

In addition to the dog and mouse plots, FIG. 6 contains CONSEQ plots of conserved regions between human and gorilla, macaque and chimp sequences for >14 kb intervals (interval 184 to 199 is shown for gorilla and macaque, and interval 228 to 244 is shown for gorilla and chimp). In the primate plots, the baseline for sequence similarity is set at 0%. Note that conformance in the gorilla and chimp is >75-80% for a large number of bases in these intervals, and that conformance for the macaque is also high, particularly when compared to the conformance of these same intervals in the mouse and dog plots below. There are, however, segments in the macaque sequence (at approximately positions 189 to 194) and in the chimp sequence (at approximately positions 234 to 237) where the conformance to both the human sequence and the gorilla sequence is low. Clearly, areas of conformance are of interest in species comparisons, as these are the regions of a genome that have been conserved over time. However, areas of nonconformance are also of interest in closely-related species or organisms. These are the regions that are most likely to contain the genetic information that differentiates the organisms.

Using techniques as described above, a comparative analysis of ˜8 Mb of orangutan, rhesus macaque, and wooley monkey DNA with orthologous human chromosome 21 sequences using high-density oligonucleotide arrays. The study focused on determining the frequency at which small genomic insertions and deletions have occurred between humans and the other primates, and on determining regional selective pressures in the human genome based on the human-chimpanzee sequence comparisons. The study identified 57 genomic rearrangements (0.2 to 8.0 kb in size) randomly distributed across the orthologous human chromosome 21 and chimpanzee chromosome 22 sequences. These rearrangements result in ˜161 kb of sequences that are present in one of the two species, but absent in the other. In the ˜8 Mb of chromosome 21 sequences compared with multiple primates, 114 genomic rearrangements (0.5 to 10 kb in size) were identified resulting in ˜414 kb of sequence differences (presence or absence) between humans and the other primates. These data suggest that a significant fraction of sequence variation between humans, apes, and old world monkeys may be the result of small genomic insertions and deletions.

Based on the neutral theory of molecular evolution, genomic intervals with low intra-species polymorphism rates reflect low regional mutational rates, and thus, should also have low interspecies fixed rates. This comparative study identified six intervals with low polymorphism rates in humans but average human-chimpanzee fixed rates. Sequencing the DNA of 10 different chimpanzees determined that these six regions have average polymorphism rates in chimpanzees. These results suggest that these six regions with decreased variation on human chromosome 21 are not the result of low regional mutation rates but likely are the result of either selective pressure or historical demographic factors.

3. Evolutionarily Conserved Sequences on Human Chromosome 21 by Comparing Human Sequence to Mouse Sequence

To identify conserved human-mouse elements, 16,580,114 bp of nonrepetitive human chromosome 21 sequence was analyzed by hybridization with orthologous mouse DNA. These human sequences represent ˜74% of the nonrepetitive chromosome 21 sequence (˜22.5 Mb). Initial analysis of the conserved human-mouse sequences consisted of classifying the elements based on whether or not they overlap known exons. In the segment of chromosome 21 analyzed, ˜1.6% (260,226 bp) of the base pairs are conserved, or which 44% correspondes to 1,141 elements in identified exons (IEs) and 56% corresponds to 2,257 conserved elements not in identified exons (NIEs). These data indicate that known exons constitute less than half of the sequences on chromosome 21 conserved between humans and mice.

To determine the global pattern of conservation on chromosome 21, the distribution of the conserved human-mouse elements in genic and nongenic intervals were determined. Genic intervals were defined as all sequences contained within 10 kb upstream to 10 kb downstream of the 216 genes annotated in the chromosome 21 sequence. Nongenic intervals were defined as all other analyzed 21 q sequences.

In the 21q nongenic intervals, ˜1% of the base pairs are conserved. These conserved base pairs comprise ˜38% of all the conserved sequences identified on chromosome 21 and ˜58% of those in NIE elements. Thus, a large fraction of the conserved sequences on human chromosome 21 exist in regions not encoding known genes.

Nonrepetitive chromosome 21 sequences (˜2.2 Mb, ˜10% of 21q) were analyzed by hybridization with both mouse and dog DNA. For these sequences ˜4.3% and ˜1.3% of the base pairs were conserved in the human-dog and human-mouse analyses, respectively. Because of the higher level of similarity at the nucleotide level between humans and dogs than between humans and mice, the human-dog analysis identified considerably more conserved elements (IEs and NIEs) than the human-mouse analysis. Furthermore, the conserved elements identified in both comparisons are usually longer in the human-dog analysis.

Based on the assumption that conserved sequences present in all three species (human/dog/mouse) are more likely to be due to active conservation rather shared ancestry, the identity of human-mouse conserved elements that are also conserved in the dog were searched for. Considering all of the human-mouse elements, 77% of the IEs and 51% of the NIEs were also identified as conserved elements in the human-dog comparison. Classifying the conserved human-mouse elements based on length and then determining the percentages that are also conserved in the dog reveals that as the length of an element increases, so does the probability that it is also detected as a conserved element in the dog. This analysis indicates that identifying evolutionarily conserved elements that are present in humans, mice, and dog is an effective approach for identifying short human-mouse elements that have been conserved due to active conservation.

4. Evolutionarily Conserved Sequences on Human Chromosome 21 by Comparing Human Sequence to Nonhuman Primates

Approximately 27 Mb of human and chimpanzee DNA were compared by hybridizing chimpanzee sequences to human chromosome 21 high-density arrays. Deletions and insertions have occurred in both the human and chimpanzee genomes and account for a large fraction of the DNA variation between the species. Some of these rearrangements map into genic regions suggesting that they may play a role in gene expression differences between humans and chimpanzees.

Numerous comparative sequence studies have demonstrated that there is more similarity at the nucleotide level between humans and chimpanzees than between humans and any other species See, J. G. Hacia, Trends Genet. 17, 637 (2001). Thus, identifying the types and extent of DNA sequence variation existing between humans and chimpanzees will be important for understanding the genetic basis of recently evolved, human specific traits (P. Gagneux, A. Varki, Mol. Phylogenet. Evol. 18, 2 (2001)). Previous comparative studies, focused on analyzing the differences between aligned human and chimpanzee sequences, have indicated that the two species are 98.4-98.77% identical at the nucleotide level (A. Fujiyama et al., Science 295, 131 (2002); B. Koop et al., Mol. Biol. Evol. 6, 580 (1989); and F. C. Chen, W. H. Li, Am. J. Hum. Genet. 68, 444 (2001)). The ˜1% of nucleotides in aligned sequences that are different between humans and chimpanzees (single nucleotide fixed differences) have, to date, been the primary focus of studies aimed at understanding the genetic differences between the two species. Although previous studies have suggested that large and small genomic rearrangements exist between human and chimpanzee DNA (E. Nickerson, D. L. Nelson, Genomics 50, 368 (1998); J. J. Yunis, O. Prakash, Science 215, 1525 (1982); S. Ueda, K. Washio, K. Kurosaki, Genomics 8, 7 (1990)), the extent and significance of DNA variation due to genomic rearrangements is poorly characterized. Furthermore, the size, chromosomal distribution, and evolutionary history of these genomic rearrangements have not yet been examined.

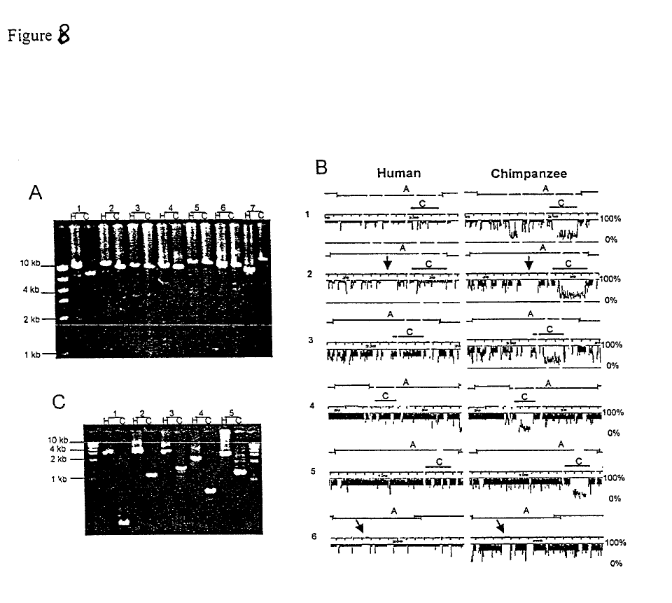

In this study human chromosome 21 was compared with the syntenic chimpanzee sequences (i.e. chimpanzee chromosome 22) to characterize the genomic rearrangements that contribute to DNA variation between the two species. A set of paired PCR primers were designed based on human sequence to amplify minimally overlapping ˜10 kb long-range PCR (LR-PCR) products spanning the entire length (˜32.4 Mb) of human chromosome 21 (N. Patil et al., Science 294, 1719 (2001). The high level of nucleotide similarity between human and chimpanzee DNA, allowed the use of this set of paired PCR primers to efficiently amplify chimpanzee chromosome 22 sequences by LR-PCR (FIG. 8A). Out of a total of 3110 paired PCR primers that successfully amplified LR-PCR products from human DNA, 2957 amplified LR-PCR products from chimpanzee DNA, resulting in the comparative analysis of ˜27 Mb of human chromosome 21 and chimpanzee chromosome 22 sequences.

LR-PCR reactions were performed using genomic chimpanzee DNA (Coriell Repository No. NG06939), and orangutan DNA (Coriell Repository No. NG12256) as previously described. The 153 paired PCR primers that initially amplified LR-PCR products from human but not chimpanzee DNA were retested. Seventy-six of these were again only successful for human DNA indicating that they specifically fail to amplify LR-PCR products from chimpanzee DNA.

The initial analysis consisted of comparing the lengths of the syntenic human and chimpanzee LR-PCR products by sizing them using gel electrophoresis (FIG. 8A). In Panel A, the lengths of syntenic human (H) and chimpanzee (C) LR-PCR products are compared by gel electrophoresis. Syntenic LR-PCR products are either the same length (6) indicating no rearrangement is present, longer in humans than in the chimpanzees (1-5) indicating the chimpanzee sequence is deleted with respect to the human sequence, or longer in the chimpanzee than in human (7) indicating that the chimpanzee sequence contains an insertion relative to the human sequence. Although the majority of the syntenic human and chimpanzee LR-PCR products are identical lengths, 33 have different sizes ranging from ˜1 kb to 8 kb as determined by inspection of the gels.

Visual inspection of the gels allowed the detection of deletions and insertions ranging from ˜1 kb to 10 kb in size. Due to the large size of the LR-PCR products (average length ˜10 kb), genomic rearrangements smaller than ˜1 kb in length result in size variations between the syntenic human and chimpanzee sequences that are too small to detect by gels. Whereas deletions in the chimpanzee genome greater than ˜10 kb in length are not detected because the LR-PCR products are not amplified (the paired PCR primers are designed based on human sequence) See Table 4. Of the syntenic LR-PCR products with different lengths, 27 were shorter and 6 were longer in the chimpanzee, suggesting that the chimpanzee DNA sequences contained deletions and insertions, respectively, relative to the human DNA sequences.

Analysis of 57 chimpanzee LR-PCR products containing rearrangements. The locations and sizes of the human-chimpanzee rearrangements are given relative to their corresponding positions on human chromosome 21. Segments=the GenBank accession number indicating which of the 106 chromosome 21 segments the rearrangement is present within. Position=the location of the insertion or deletion in the segment. For chimpanzee LR-PCR products containing insertions and deletions that were only detected by size variations on gels, the position indicates the starting location of the LR-PCR product in the segment. For deletions detected by the human chimpanzee comparative 21 q data, the position indicates the exact location (rounded to the nearest 1-kb) of the rearrangement. I/D=insertion/deletion. Deletions that are detectable only by LR-PCR product size variations on gels (D₁) most likely are composed of repetitive elements which are not tiled on the high-density arrays. Size (kb)=the size of the rearrangement. Gel=the rearrangement in the LR-PCR product was detectable (Y) or not detectable (N) by size variations on gels. Hyb=the rearrangement was detectable (Y) or not detectable (N) by inspection of the 21q array data.

TABLE 4

| Segment | Position | I/D | Size (kb) | Gel | Hyb |
|---|---|---|---|---|---|
| 2 AL163202 | 292 kb | I | 7 | Y | N |
| 2 AL163202 | 301 kb | I | 5 | Y | N |
| 3 AL163203 | 124 kb | D | 7 | Y | Y |
| 3 AL163203 | 158 kb | D1 | 3 | Y | N |
| 3 AL163203 | 263 kb | I | 5 | Y | N |
| 4 AL163204 | 18 kb | D | 2 | N | Y |
| 5 AP001660 | 45 kb | D | 2 | Y | Y |
| 8 AL163208 | 218 kb | D | 5 | Y | Y |
| 10 AL163210 | 161 kb | D | 2 | N | Y |
| 12 AP001667 | 278 kb | I | 5 | Y | N |
| 17 AP001672 | 86 kb | I | 1 | Y | N |
| 25 AP001680 | 228 kb | D | 0.4 | N | Y |
| 26 AP001681 | 305 kb | D | 0.4 | N | Y |
| 28 AP001683 | 157 kb | D | 0.3 | N | Y |
| 28 AP001683 | 250 kb | D | 1.5 | N | Y |
| 31 AP001686 | 119 kb | D | 1 | N | Y |
| 32 AP001687 | 325 kb | D | 1.5 | Y | Y |
| 32 AP001687 | 330 kb | D | 3 | Y | Y |
| 33 AP001688 | 9 kb | D | 3 | Y | Y |
| 33 AP001688 | 41 kb | D | 2 | N | Y |
| 33 AP001688 | 185 kb | D | 4 | Y | Y |
| 33 AP001688 | 197 kb | D | 7 | Y | Y |
| 35 AP001690 | 262 kb | D | 8 | Y | Y |
| 35 AP001690 | 326 kb | D | 3 | Y | Y |
| 36 AP001691 | 28 kb | D | 3 | Y | Y |
| 37 AP001692 | 205 kb | D | 5 | Y | Y |
| 39 AP001694 | 325 kb | D1 | 5 | Y | N |
| 40 AP001695 | 313 kb | D | 1 | N | Y |
| 40 AP001695 | 321 kb | D | 1 | N | Y |
| 41 AP001696 | 156 kb | D | 0.5 | N | Y |
| 52 AP001707 | 172 kb | D | 1 | N | Y |
| 53 AP001708 | 102 kb | D | 2 | N | Y |
| 53 AP001708 | 144 kb | D | 6 | Y | Y |
| 54 AP001709 | 159 kb | D1 | 1 | Y | N |
| 57 AP001712 | 229 kb | D | 2 | N | Y |
| 61 AP001716 | 79 kb | D | 3 | Y | Y |
| 61 AP001716 | 237 kb | D | 3 | N | Y |
| 61 AP001716 | 326 kb | D | 2 | N | Y |
| 64 AP001719 | 298 kb | D | 2 | N | Y |
| 70 AL163270 | 51 kb | D | 6.5 | Y | Y |
| 71 AL163271 | 234 kb | D | 2.8 | Y | Y |
| 76 AL163276 | 51 kb | D | 7 | Y | Y |
| 76 AL163276 | 130 kb | D | 0.5 | N | Y |
| 77 AL163277 | 24 kb | D | 2.5 | N | Y |
| 77 AL163277 | 125 kb | D | 2.5 | Y | Y |
| 77 AL163277 | 226 kb | D | 2 | Y | Y |
| 77 AL163277 | 288 kb | D | 2 | Y | Y |
| 78 AL163278 | 227 kb | D | 1.8 | Y | Y |
| 82 AL163282 | 207 kb | I | 5 | Y | N |
| 84 AL163284 | 250 kb | D | 5 | Y | Y |
| 96 AP001751 | 89 kb | D | 1 | N | Y |
| 97 AP001752 | 158 kb | D | 2 | Y | Y |
| 100 AL163300 | 229 kb | D | 1 | Y | Y |
| 101 AL163301 | 138 kb | D | 2 | N | Y |
| 104 AP001759 | 29 kb | D | 0.2 | N | Y |
| 104 AP001759 | 111 kb | D | 0.5 | N | Y |
| 104 AP001759 | 141 kb | D | 0.2 | N | Y |

To determine if the shorter length chimpanzee LR-PCR products contain a single localized deletion or numerous small dispersed deletions, the amplified chimpanzee DNA was examined using a series of human chromosome 21 high-density arrays.

High-density oligonucleotide arrays have proven to be a rapid approach for comparing human sequences with the DNA of other mammalian species. The 21q high density arrays consist of a series of 8 wafer designs, on which each of the unique chromosome 21 bases is interrogated by 8 unique oligonucleotides (25-mers) as previously described. Because only unique human sequences are tiled on the 21q arrays, sequence deletions solely encompassing interspersed repeats are not detected in the comparative 21q array data. Likewise, insertions represent DNA present in chimpanzees but not in humans, and thus, this class of rearrangements is also not detected by analysis of the comparative 21q array data.

The chimpanzee LR-PCR products were pooled based on the syntenic human chromosome 21 sequences represented on each of the 21q high-density arrays, and hybridized as a single reaction. Analysis of the comparative human-chimpanzee 21q array data revealed that the majority of chimpanzee LR-PCR products that are shorter length than their syntenic human counterparts contain a single localized deletion.

In FIG. 8, Panel B, the human and chimpanzee LCR-PCR products shown in (A) were hybridized to the 21q arrays and their percent conformances (vertical axis), which is a measure of their similarity , were plotted relative to their position in the human reference sequence (horizontal axis). Each tick mark in the scale represents a 1 kb interval. The sequence positions of the PCR products in (A), and (C) are indicated by horizontal lines. The overlap of the LR-PCR products in (A) with neighboring chromosome 21 LR-PCR products is shown. For the chimpanzee analyses, the sharp drop in conformance values (yellow circles) indicates the positions of the deleted sequences in the LR-PCR products 1-5 in (A). Sequences with absent conformance information (black arrows) correspond to interspersed repeats (short gold rectangles), which were not tiled on the 21 q arrays. There are two localized deletions in LR-PCR product #1 and for LR-PCR product #5 the variation in the sizes of the human and chimpanzee bands are not detectable on the gel.

Four of the deletions observed by variations in the size of syntenic human and chimpanzee LR-PCR products on gels were not detected by analysis of the comparative 21q array data, and likely are composed of interspersed repeats. One of the chimpanzee LR-PCR products contains two localized deletions (FIG. 8).

Moreover, the deletions largely result in the loss of sequences that are unique in the human genome. These results are the first direct evidence that the human genome contains intervals several kilobases in length and comprised of unique sequences which are not present in the syntenic regions of the chimpanzee genome. See, J. G. Hacia et al., Nat Genet. 18, 155 (1998); J. G. Hacia et al., Nat Genet. 22, 119 (1999); and K. A Frazer et al., Genome Res. 11, 1651 (2001).

The comparative human-chimpanzee 21q array data was next examined to determine if additional deletions in the amplified chimpanzee sequences could be identified. The deletion signature in the array data—a sharp decrease in the conformance rate within the boundaries of an amplified chimpanzee LR-PCR product (FIG. 8B)—was searched for and 24 such intervals were found (˜0.2-3.0 kb in length) (See Table 4).

Labeled chimpanzee sequences were hybridized with the human 21q arrays, if the probe complementary to the human reference sequence had greater fluorescent intensity than the corresponding noncomplementary probes, the nucleotide under interrogation was referred to as conforming to the human reference sequence. To calculate the “conformance rate” a 30-nt length windows was examined and the conformance of the individual nucleotides was averaged. For example, if 27 of the 30 nucleotides conformed to the human reference sequence, the window would have a 90% conformance rate.

To demonstrate that these intervals of low conformance on the human chromosome 21 arrays correspond to sequence deletions on chimpanzee chromosome 22, paired PCR primers to sequences bordering five of the intervals were designed and compared the lengths of PCR products amplified from human and chimpanzee genomic DNA. As shown in FIG. 5C, paired PCR primers designed to the external boundaries of the deletions in LRPCR products 1-5 in (A) as shown in (B) were used to amplify human and chimpanzee DNA. The lengths of the human versus chimpanzee PCR products

In all cases, the syntenic human PCR product was longer then the chimpanzee PCR product by the approximate base pair amount predicted by the comparative 21q array data. These data indicate that comparative analysis of human and chimpanzee DNA using high-density arrays is an effective method for identifying intervals in the human genome containing unique sequences that are missing in the syntenic regions of the chimpanzee genome.

In the ˜27 Mb segment of chromosome 21 analyzed, small genomic rearrangements account for ˜0.6% (161 kb) base pair differences between the syntenic human and chimpanzee sequences, of which ˜82% corresponds to 51 sequence deletions and ˜18% corresponds to 6 sequence insertions in the chimpanzee DNA (See Table 4). The observation that deletions are more prevalent than insertions is at least in part due to an ascertainment bias based on the fact that insertions are only detectable by variation in the size of the LR-PCR products on gels whereas deletions are identified by size variations on gels and the comparative 21q array data. Rearrangements smaller and larger in size than the detectable range in this study (0.2 to 10.0 kb) are likely to also be present, and thus, the data represent the minimal amount of base pair differences between the syntenic human chromosome 21 and chimpanzee chromosome 22 sequences due to insertions and deletions. These results suggest that small genomic rearrangements are responsible for a significant fraction of the DNA variation between humans and chimpanzees, accounting for ˜50% as much DNA variation as single nucleotide fixed differences. Inspection of the human sequences at the boundaries of the rearrangements revealed that both unique as well as a variety of repetitive sequences are present. These data neither implicate a particular class of sequences nor suggest an obvious mechanism that gives raise to these rearrangements.

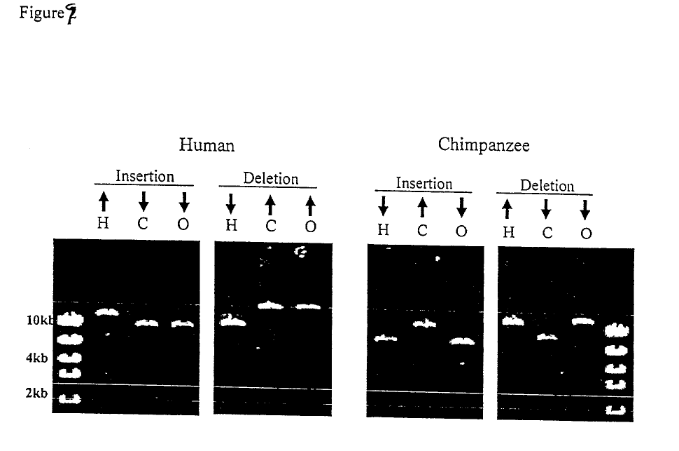

To elucidate whether the 57 small genomic rearrangements responsible for DNA variation between humans and chimpanzees are the result of deletions and insertions occurring predominately in one or the other of these primates, the orangutan was used as an outgroup. For 16 of the human-chimpanzee DNA rearrangements, the relative sizes of the corresponding syntenic human, chimpanzee, and orangutan LR-PCR products were examined, and thereby it was ascertained for each of these rearrangements whether it occurred in the human genome (the chimpanzee and orangutan LR-PCR products are the same) or the chimpanzee genome (the human and orangutan LR-PCR products are the same) (FIG. 9). If the human LR-PCR product is larger or smaller than the syntenic chimpanzee and orangutan LRPCR products, then an insertion or deletion occurred, respectively, in the human genome. Whereas if the chimpanzee LR-PCR product is larger or small than the syntenic human and orangutan LR-PCR products, then an insertion or deletion occurred, respectively, in the chimpanzee genome.

Of the 16 rearrangements that were examined, 6 occurred in the human genome and 10 occurred in the chimpanzee genome (See Table 5). These data indicate that small genomic deletions and insertions have occurred in both the human and chimpanzee genomes.

Table 5 shows an analysis of 16 syntenic human, chimpanzee, and orangutan LRPCR products. Segments=the GenBank accession number indicating which of the 106 chromosome 21 segments the rearrangement is present within. Position=the location of the insertion or deletion in the segment. For LR-PCR products containing insertions and deletions that were only detected by size variations on gels, the position indicates the starting location of the LR-PCR product in the segment. For deletions detected by the human-chimpanzee and human-orangutan comparative 21q data, the position indicates the exact location (rounded to the nearest 1-kb) of the rearrangement. I/D=insertion/deletion. Size (kb)=the size of the rearrangement. Gel=the rearrangement in the LR-PCR product was detectable (Y) or not detectable (N) by size variations on gels. Hyb=the rearrangement was detectable (Y) or not detectable (N) by inspection of the 21q array data. (+)=the nonhuman primate LR-PCR product was a different size than the syntenic human LR-PCR product. (−)=the nonhuman primate LR-PCR product was the same size as the syntenic human LR-PCR product.

TABLE 5

| Segment | Position | Size (kb) | I/D | Gel | Hyb | Chimpanzee | Orangutan |
|---|---|---|---|---|---|---|---|
| 2 AL163202 | 292 kb | 7 | I | Y | N | + | + |
| 2 AL163202 | 301 kb | 5 | I | Y | N | + | + |
| 3 AL163203 | 158 kb | 3 | D1 | Y | N | + | + |
| 4 AL163204 | 18 kb | 2 | D | N | Y | + | + |
| 5 AP001660 | 45 kb | 2 | D | Y | Y | + | − |
| 8 AL163208 | 218 kb | 5 | D | Y | Y | + | − |
| 10 AL163210 | 161 kb | 2 | D | N | Y | + | + |
| 12 AP001667 | 278 kb | 5 | I | Y | N | + | − |
| 70 AL163270 | 51 kb | 6.5 | D | Y | Y | + | + |
| 71 AL163271 | 234 kb | 2.8 | D | Y | Y | + | − |
| 76 AL163276 | 51 kb | 7 | D | Y | Y | + | − |
| 76 AL163276 | 130 kb | 0.5 | D | N | Y | + | − |
| 77 AL163277 | 24 kb | 2.5 | D | N | Y | + | − |
| 77 AL163277 | 125 kb | 2.5 | D | Y | Y | + | − |
| 77 AL163277 | 288 kb | 2 | D | Y | Y | + | − |
| 78 AL163278 | 227 kb | 1.8 | D | Y | Y | + | − |

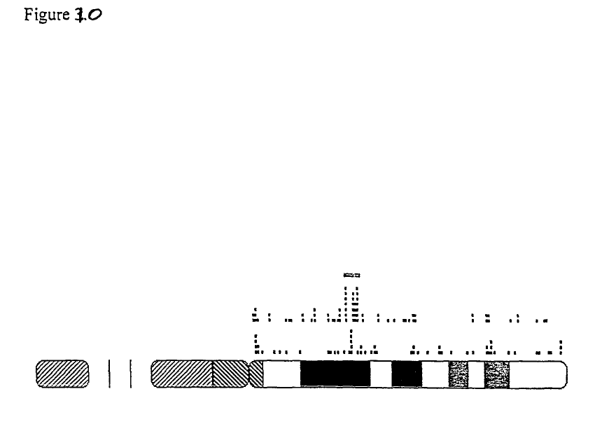

To determine the spatial distribution of the human-chimpanzee rearrangements, chromosome 21 was divided into 132 adjacent 250 kb intervals and the number of genomic rearrangements mapping into each interval were determined (FIG. 10). A statistical analysis revealed that the rearrangements are uniformly distributed, except for one 250 kb interval which contains an increased number of rearrangements (p<0.01).

To determine the expected distribution of the 57 rearrangements 10,000 simulations were performed, and for each, the number of rearrangements mapping into each of the 250 kb intervals was calculated. In the data, there is one 250 kb interval containing 5 rearrangements. I n the simulations, the probability of seeing 5 or more rearrangements per interval was about 0.01. A similar analysis for the distribution of the 76 human-specific LR-PCR products was performed. Two 250 kb intervals containing 7 human-specific LR-PCR products were observed, the probability of seeing this in the stimulations was 0.0005.

To further investigate the spatial distribution of diverged human-chimpanzee sequences, the distribution of 76 paired PCR primers that amplified LR-PCR products from human but not chimpanzee DNA were examined. The fact that these paired PCR primers (designed based on human sequence) specifically fail to amplify LR-PCR products from chimpanzee DNA suggests that their corresponding sequences have either been rearranged or have significantly diverged in the chimpanzee genome. Thus, looking at the distribution of these chimpanzee specific LR-PCR failures is an indirect way of identifying regions containing either a genomic rearrangement or high sequence divergence. In agreement with the distribution analysis of the genomic rearrangements, the paired PCR primers corresponding to the chimpanzee specific LR-PCR failures are uniformly distributed in the 250 kb intervals on chromosome 21, except for two intervals that contain increased LR-PCR failures (p<0.0005) (FIG. 9). Interestingly, the three 250 kb intervals that were identified, which contain either an increased number of rearrangements and/or an increased amount of sequence divergence, are clustered within an ˜1 Mb gene poor region on chromosome 21 (See, M. Hattori et al., Nature 405, 311 (2000).) A previous human-chimpanzee DNA comparison based on a sequence-tagged site (STS) approach also reported clustering of rearrangements in this same region of chromosome 21. The data described herein indicates that the majority of small genomic rearrangements are uniformly distributed but that a gene poor region on chromosome 21 exists that contains an increased number of rearrangements and/or a greater amount of sequence divergence than is expected by chance.

In addition to an essentially uniform distribution along the length of chromosome 21, it was observed that these deletions occur at relatively equal frequencies in genic and nongenic intervals. Genic intervals (˜13.0 Mb) were defined as all sequences contained within 10 kb upstream to 10 kb downstream of the 215 annotated genes, and nongenic sequences (˜20.9 Mb) were defined as all other sequences on human chromosome 21. Twenty of the rearrangements mapped into genic intervals, and 37 mapped into nongenic intervals.

Of the 20 rearrangements mapping into genic intervals, 13 were located in the introns of 12 genes (USP25, NCAM2, PRED16, APP, PRED29, PRED33, IL10RB, KCNJ15, DSCAM, ERG, C21orf1, ADARB1) and 7 were located within 10 kb upstream or downstream of 7 genes (CLDN17, KCNE2, C21orf5, PRED47, CSTB, COL6A1, and COL6A2).

These data indicate that small genomic rearrangements are frequently located in the vicinity of genes, and thus, they may be partially responsible for the differential expression of certain genes between humans and chimpanzees. It has long been postulated that mutations of regulatory elements controlling the expression of genes will be responsible for the major biological differences between humans and chimpanzees. See, M. C. King, A. C. Wilson, Science 188, 107 (1975). The data demonstrates that deletions and insertions have resulted in humans containing sequences which are unique, several kilobases in length, and located in genic regions, that are not present in chimpanzees. These rearrangements provide an excellent starting point for the study of gene expression differences in humans and chimpanzees as part of an effort to identify the genetic differences responsible for the biological, physiological and behavior differences between these species.

It is to be understood that the above description is intended to be illustrative and not restrictive. Many embodiments will be apparent to those skilled in the art upon reviewing the above description. The scope of the invention should, therefore, be determined not with reference to the above description, but should instead be determined with reference to the appended claims, along with the full scope of equivalents to which such claims are entitled.

What is claimed is:

1. A method for identifying evolutionarily conserved or divergent sequences of a human, comprising:
collecting a plurality of hybridization intensities wherein each of said intensities reflects the hybridization of one of a plurality of probes from a first nucleic acid sequence from a first organism to a sample nucleic acid from a second organism, wherein said probes are complementary and non-complementary to a known nucleic acid sequence from said first organism, wherein said probes are arrayed on a substrate and wherein each detection probe is at a known location on said substrate;
identifying bases of said plurality of probes according to said hybridization intensities; and
calculating an identity index between said first nucleic acid sequence from said first organism and said sample nucleic acid from said second organism.

2. The method of claim 1, wherein said detection probes are sets of four probes where one probe of said probe set is perfectly complementary to said known nucleic acid sequence and three probes of said probe set are non-complementary to said known nucleic acid sequence.

3. The method of claim 2, wherein said non-complementary probes differ from said known nucleic acid sequence by one base.

4. The method of claim 3, wherein said one base is a base located at or near a central position of said probe.

5. The method of claim 1, wherein said sample nucleic acids are nucleic acids which have been amplified by the polymerase chain reaction.

6. The method of claim 1, wherein said identity index is calculated by determining a percentage of similarity between sub-regions of said nucleic acids from said first organism and said nucleic acids from said second organism

7. The method of claim 6, wherein said sub-regions are overlapping, moving windows of base pairs across said nucleic acid sequence from a first organism.

8. The method of claim 7, wherein said windows are between about 20 base pairs and 150 base pairs.

9. The method of claim 7, wherein said overlap of said windows is between about 5 base pairs and about 75 base pairs.

10. A method for screening for functional sequences in a genome of a first organism, comprising:
providing a substrate having a plurality of detection probes, wherein each detection probe is at a known location, and wherein at least one of said detection probes is complementary to a known nucleic acid sequence in the genome from said first organism and at least one of said detection probes is non-complementary to a known nucleic acid sequence in the genome from said first organism;
contacting at least one sample nucleic acid from a second organism with said substrate, where said second organism diverged evolutionarily from said first organism between about 60 million years ago and about 120 million years ago, and where said contacting is performed under conditions wherein when said at least one sample nucleic acid is substantially complementary to a detection probe said at least one sample nucleic acid will preferentially hybridize to a detection probe to which it is most complementary, resulting in at least one hybridized detection probe;
determining a location of said at least one hybridized detection probe; and
identifying sequences of said at least one hybridized detection probe by referring to the location of said at least one hybridized detection probe; wherein when said sequence of said at least one hybridized detection probe is the same as a sequence complementary to said known nucleic acid sequence from said first organism, there is sequence similarity between nucleic acids from said first organism and said second organism, and regions in said nucleic acids of said first organism where there is sequence similarity with said nucleic acids from said second organism are candidate functional regions in said nucleic acids of said first organism.

11. The method of claim 10, wherein said detection probes are sets of four probes where one probe of said probe set is perfectly complementary to said known nucleic acid sequence and three probes of said probe set are non-complementary to said known nucleic acid sequence.

12. The method of claim 11, wherein said non-complementary probes differ from said known nucleic acid sequence by one base.

13. The method of claim 12, wherein said one base is a base located at or near a central position of said probe.

14. The method of claim 10, wherein said probes are at least 18 bases long.

15. The method of claim 10, wherein said sample nucleic acids are nucleic acids which have been amplified by the polymerase chain reaction.

16. The method of claim 10, further comprising the step of calculating an identity index between sub-regions of said nucleic acids from said first organism and said nucleic acids from said second organism.

17. The method of claim 16, wherein said identity index is calculated by determining a percentage of similarity between sub-regions of said nucleic acids from said first organism and said nucleic acids from said second organism.

18. The method of claim 17, wherein said sub-regions are overlapping, moving windows of base pairs across said nucleic acid sequence from a first organism.

19. The method of claim 18, wherein said windows are between about 20 base pairs and 150 base pairs.

20. The method of claim 18, wherein said overlap of said windows is between about 5 base pairs and about 75 base pairs.
